(19) United States
(12) Patent Application Publication
(10) Pub. No.: US 2023/0333107 A1
(43) Pub. Date: Oct. 19, 2023

(54) METHOD OF IDENTIFYING OR CHARACTERISING AN IMMUNE RESPONSE IN A SUBJECT

(71) Applicant: THE BINDING SITE GROUP LIMITED, Birmingham West Midlands (GB)

(72) Inventors: Stephen HARDING, Birmingham West Midlands (GB); David BARNIDGE, Rochester, MN (US); Charles DE ROHAN, Birmingham West Midlands (GB); Richard HUGHES, Birmingham West Midlands (GB); Sabah PASHA, Birmingham West Midlands (GB); Simon NORTH, Birmingham West Midlands (GB); Roshani PATEL, Birmingham West Midlands (GB); Gregg WALLIS, Birmingham West Midlands (GB)

(21) Appl. No.: 17/927,178

(22) PCT Filed: May 20, 2021
(86) PCT No.: PCT/GB2021/051227

§ 371 Date: Nov. 22, 2022

Related U.S. Application Data
(60) Provisional application No. 63/028,887, filed on May 22, 2020.

(30) Foreign Application Priority Data
Jun. 11, 2020 (GB) 2008887.8

(51) Int. Cl. G01N 33/569; G01N 33/68

(52) U.S. Cl. CPC G01N 33/56983; G01N 33/6851; G01N 2333/165; G01N 2469/20

(57) ABSTRACT

This invention provides a method of identifying or characterising an immune response in a subject comprising: (a) contacting a sample containing immunoglobulins from the subject with at least one antigen immobilised on a support; (b) washing unbound, non-antigen specific immunoglobulins from the support to leave antigen-specific immunoglobulins bound to the antigen on the support; (c) optionally eluting the antigen-specific immunoglobulins from the antigen on the support; and (d) subjecting the antigen-specific immunoglobulins to mass spectrometry to identify two or more different antigen specific immunoglobulin classes, subclasses and/or light chain types.

The invention provides a method of identifying and quantifying an immune response in a subject by using for example tryptic digestion fragments to quantify antigen-specific immunoglobulins by mass spectrometry. Also provided are computer implemented methods and assay kits for use in the method. The methods are especially useful for characterising the immune response for example against viral, fungal and bacterial infections, autoantigens and other immune responses, including the characterisation of vaccine candidates.

Human immunoglobulins contain two identical heavy chain polypeptides and two identical light chain polypeptides bound together by disulphide bonds. There are two different light chain isotypes (kappa and lambda) and five different heavy chain isotypes (IgG, IgA, IgM, IgD and IgE).

IgG provides the majority of antibody-based immunity against invading pathogens.

IgA tends to be found in mucosal areas, such as the gut, respiratory tract and urogenital tract and often prevents colonisation by pathogens. It is also found in saliva, tears and breast milk. It is usually found as dimers connected by a so-called J-chain peptide.

IgM is expressed on the surface of B-cells as a monomer and in a secreted form as a pentamer and eliminates pathogens in the early stages of B-cell-mediated (humoral) immunity. The pentamer of IgM is connected together via J-chain peptides.

IgD is an antibody that makes up about 1% of proteins in the plasma membrane of immature B-lymphocytes and makes up approximately 0.25% of immunoglobulins in serum

IgE is found in mammals and is often involved in in immunity to parasites, such as helminths and also has a role in many types of hypersensitivity to allergens.

IgG has four subclasses, IgG1, IgG2, IgG3 and IgG4. Those subclasses are differentiated on the basis of the size of the hinge region, position of interchain disulphide bonds, amino acid sequence of the constant region, and molecular weight. The subclasses also differ in their ability to activate complement and their binding to Fc receptors.

IgG1 comprises 60-65% of the main subclass IgG and is predominantly responsible for thymus-mediated immune response (also known as a T-cell mediated immune response) against protein and polypeptide antigens. It is also involved in opsonisation and activation of the complement cascade.

IgG2 comprises 20-25% of the main subclass and is the prevalent immune response against carbohydrate and polysaccharide antigens. Among all IgG isotype deficiencies, a deficiency in IgG2 is the most common and is associated with recurring airway and respiratory infections in infants.

IgG3 comprises around 5-10% of the total IgG and plays a major role in immune responses against protein or polypeptide antigens.

IgG4 comprises usually less than 4% of total IgG. IgG4 does not predominantly bind to polysaccharides. Elevated IgG4 levels are seen in IgG4 related disease (RD), this is an immune-mediated and chronic fibro-inflammatory condition with a characteristic histopathological appearance. Quantification of serum IgG4 is included in all IgG4-RD diagnostic guidelines available to-date, including type 1 autoimmune pancreatitis (AIP) which is the pancreatic manifestation of IgG4-RD. although, elevated IgG4 is not observed in all IgG4-RD patients. Recent studies have shown that elevated serum levels of IgG4 are found in patients suffering from sclerosing pancreatitis, cholangitis and interstitial pneumonia caused by infiltrating IgG positive plasma cells. The precise role of IgG4 is still mostly unknown.

Table 1 shows the comparison of the IgG antibody isotypes or subclasses in humans. This includes a summary of the response of the different subclasses to different antigens. It also includes a summary of their complement activation and binding to Fc receptors involved in antigen recognition. The latter are located at the membrane of for example B lymphocytes, killer cells, macrophages, neutrophils and mast cells. Such receptors recognise the Fc fragment of antibodies.

IgA, which is also referred to sIgA in its secretory form (as a dimer) has two subclasses, IgA1 and IgA2. IgA is a poor activator of the complement system and opsonises only weakly. Whilst IgA1 predominates in serum (approximately 80%), IgA2 percentages are higher in secretions than in serum (approximately 35% in secretions). IgA1 is the predominant IgA subclass found in serum, with most lymphoid tissues having a majority of IgA1-producing cells. In one allotype, IgA2m(1), the heavy and light chains are not linked with disulphide, but with non-covalent bonds. In secretory lymphoid tissues, such as gut-associated lymphoid tissue, the share of IgA2 production is larger than in non-secretory lymphoid organs, such as the spleen and peripheral lymph nodes. Polysaccharides antigens tend to induce more IgA2 than protein antigens.

There are additionally two antibody isotypes not found in mammals. IgY is found in birds and reptiles and is related to mammalian IgG. IgD is found in sharks and skates, and is related to mammalian IgD.

The difference in the ability of immunoglobulin subclasses to activate complement and mediate antibody cell cytotoxicity, means that variations in the immunoglobulin type and subclass which are activated can have a positive or negative effect on immune responses, such as to infection. For example, IgG3 has been associated with an enhanced control or protection against a range of intracellular bacteria, parasites and viruses. IgG3 antibodies are potent mediators of effect or functions, including enhanced ADCC, opsonophagocytosis, complement activation and neutralisation, compared with other IgG subclasses. It is therefore believed that future antibody-based therapeutics and vaccines should consider utilising IgG3, based on features of enhanced functional capacity. Investigating the impact of glycosylation patterns and allotypes on IgG3 function may expand our understanding of IgG3 responses and their therapeutic potential.

IgG3 comprises only a minor fraction of IgG and has remained relatively understudied until recent years. Recent studies underscore the importance of IgG3 effector functions against a range of pathogens and have provided approaches to overcome IgG3-associated limitations, such as allotype-dependent short antibody half-life and excessive pro-inflammatory activation. Understanding the molecular and functional properties of IgG3, and other immunoglobulin subclasses may therefore facilitate the development of improved antibody-based immunotherapies and immunotherapies and vaccines against infectious diseases.

The detection of immunoglobulins and their subclasses is known in the art. However, nephelometry and turbidimetry approaches will measure overall immunoglobulin quantities, rather than antigen-specific quantities. ELISA methods for antigen specific quantities utilise immunoglobulin subclass specific antibodies for detection. These antibodies will have different avidity for each class or subclass. Accordingly, a comparison for between, for example, IgG subclass or IgA subclass assays may not be accurate. Furthermore, they will have different calibration curves and other than relatively few exceptions there is a paucity of international standards to measure antibody responses to different pathogenic antigens, meaning between assay result comparison is not possible. Because of the difference in the apparent amounts observed, this prevents the possibility of any ratio or comparison between the different classes, subclasses or light chain types in a single assay

Ladwig, P.M. et al., Clinical Chemistry (2014), Volume 16, pages 1080-1088 discusses the absolute quantification of IgG subclasses using mass spectrometry. They describe the combining of serum with stable isotope labelled internal peptide standards and intact purified horse IgG. Samples with denatured, reduced, alkylated and digested with a peptidase, such as trypsin. They then analysed the digested serum by LC-MS/MS for IgG subclasses 1-4 and total IgG. They were able to demonstrate that it was possible to quantify total IgG and IgG subclasses 1, 2, 3 and 4 using LC-MS/MS. The Isotyping of heavy chain and light chain isotypes is also described in WO 2015/154052, incorporated herein in its entirety. This describes the detection of such isotypes by mass spectrometry (MS).

The inventors have realised that having a single method to simultaneously and quantitatively profile the antibody response, for example before, during, or after infection, will allow better clinical intervention. They realised that the enrichment of antigen or disease-specific antibodies by solid-phase capture, followed by analysis by mass spectrometry allows automated analysis of samples to be carried out, without the disadvantages of, for example, ELISA-based assays. This is expected to improve the ability of clinicians to manage a subject’s immune response to a disease, and to allow the investigation of immune responses to antigens in general, with a possible improvement of the production of vaccines and monoclonal antibodies.

The invention provides a method of identifying or characterising an immune response in a subject, comprising:
(a) contacting a sample containing immunoglobulins from the subject with at least one antigen immobilised on a support;
(b) washing unbound, non-antigen specific immunoglobulins from the support to leave antigen-specific immunoglobulins bound to the antigen on the support;
(c) optionally eluting the antigen-specific immunoglobulins from the antigen on support; and
(d) subjecting the antigen-specific immunoglobulins to mass spectrometry to identify two or more different antigen specific immunoglobulin classes, subclasses and/or light chain isotypes. The immunoglobulins may be monoclonal or polyclonal in nature

Typically, two or more different antigens are provided. These may be provided on different supports. Accordingly, for example, the sample may be separated into different aliquots, with each aliquot being contacted with a different antigen on a different support. Alternatively, the sample may be contacted with antigens on different portions of the support.

The support may be any support, generally known in the art. These include, agarose, cellulose, glass, paramagnetic or magnetic (such as ferrous) or polystyrene beads. The support may also be, for example, a well on a microtiter plate, or indeed the target for use in, for example MALDI-TOF MS. Typically, paramagnetic beads or a MALDI-TOF target are used. Paramagnetic beads are especially useful because they are easily separated from fluids surrounding the beads by means of a magnet. The antigens are typically covalently attached to the beads using chemistry generally well known in the art. These include, for example, use of commercially available streptavidin-coated beads, and attaching the antigen via a biotin moiety attached to the antigen. There are a number of other surface coatings for beads which are commercially available to facilitate the binding of antigens to such beads, including a variety of amino, carboxy, alkyl, thiol, epoxy, hydrazine and tosyl groups. Also used, for example, is ConA, to bind, for example, sugars on carbohydrate-binding proteins and RHO1D4, which are efficient in the purification of membrane proteins.

The unbound non-antigen specific immunoglobulins are optionally washed off with a suitable buffer or other wash. Again, such washes are generally known in the art. Such washers include, for example, phosphate buffered saline containing, for example, 0.1% TWEEN. Two or more washes may be used to ensure the removal of non-antigen specific immunoglobulins.

The antigen-specific immunoglobulins may be eluted off the antigen using a suitable elution buffer, such as an acid buffer. The invention also allows antibodies of different specificities to be eluted from the antigen, for example, by using different concentrations of salt or other conditions in the elution washes. Therefore, after the non-specific binding markers has been removed, it may be possible to remove immunoglobulins having lower antigen specificity, with a first elution buffer, followed by removing those with having a higher antigen binding specificity using a second elution buffer. This may be useful where, for example, the antigen of interest has different parts to the antigen having different levels of antigenicity to the immune system of the subject.

Alternatively if, for example the antigen is bound to a mass spectrometry target such as a MALDI-TOF target, then the antigen bound immunoglobulins may be ionised and analysed directly from the target.

The antigen-specific immunoglobulins may be subjected to a proteolytic digestion prior to subjecting the digested antigen-specific immunoglobulins to mass spectrometry. Typically, the enzyme used for the digestion is an endopeptidase, which fragments the immunoglobulins within the chain of the immunoglobulin, rather than one end of the chain of the immunoglobulin. Examples of suitable endopeptidase, include trypsin. Ludwig, et al. supra, for example, uses trypsin in combination with water and ammonium bicarbonate to perform a tryptic digest of immunoglobulins, prior to undertaking mass spectrometry.

The use of LC-MS/MS in combination with tryptic digests has also been demonstrated in Gugten, Clinical Chemistry, Volume 64, 735-742 (2018). This demonstrated that immune-based diagnostic kits, occasionally produces suspected analytical errors in patients, for example with increases in total IgG4. This resulted in errors in the calculation of the total IgG concentration. Using LC-MS/MS, in combination with a calibrator, the apparent discrepancies were able to be reduced. The assay was again used to detect the total amounts of non-antigen specific immunoglobulins and immunoglobulins subclasses in subjects, for example, the assessment of immune deficiency and IgG4-related disease.

The use of LC-MS/MS in combination of tryptic digests has also been demonstrated by Remily-Wood et al., Proteomics Clin Appl. 2014 October; 8(0): 783-795. The authors described the use of isotype and subclass specific tryptic peptides to identify and monitor monoclonal immunoglobulins in serum. The isotype and subclass specific tryptic peptides used in this study are shown in Table 2.

The table shows a list of isotopically labelled peptides that can be synthesised, the labelled amino acid is underlined and the asterisks indicate where there is a conservative amino acid replacement. The Table shows protein, peptide sequence (and whether it is isotope labelled or a structural analogue created by a conservative single amino acid replacement) and transitions

Alternatively, at least a portion of the antigen specific immunoglobulins are not subject to a proteolytic digestion prior to being subjected to mass spectrometry. That is, the intact heavy chain and/or light chain may be detected by mass spectrometry.

Typically, at least a portion of the antigen specific immunoglobulins may be dissociated with at least one reducing agent to separate light chains down to heavy chains prior to subjecting the separated immunoglobulins to mass spectrometry. This may occur, for example, prior to proteolytic digestion where used. This, again, is generally known in the art, for example, as shown in WO 2015/154052 and Ladwig, et al., both incorporated herein by reference in their entirety.

The antibodies in a sample are bound to the antigen and are then separated into their respective classes, subclasses or types. This means that the relative peak for each class, subclass or type detected can be compared to each other to allow the relative amount of the classes, subclasses or light chain types in a sample to be determined. This removes some of the ambiguity often seen for ELISA-type assays.

Alternatively, an internal digestion control may be used. The control may, for example, be an immunoglobulin from another, different, species of animal to the subject. For example horse IgG was used by Ladwig et al supra, because horse immunoglobulin tryptic peptides have a different amino acid sequence and therefore a different mass than the human immunoglobulin tryptic peptide equivalent. This allows the control to be distinguished from the immunoglobulin peaks and to be used to determine completion of tryptic digestion. A pre-determined amount of a synthesized stable isotope labelled internal standard peptide may be added, for example, prior to the mass spectrometry. The internal standard peptide has the exact same amino acid sequence as isotype and subclass specific tryptic peptides but is heavier due to the addition of stable isotopes such as C13 and N15 in the amino acids used to synthesize the peptide. The chemical properties of the internal standard peptide are the same as the native tryptic peptide from the Ig but its mass is different and can therefore be monitored alongside the native peptide in the mass spectrometer.

Absolute quantification is accomplished by reporting the ratio of the abundance of the internal standard peptide to the abundance of the native peptide in an unknown sample as observed in a mass spectrometer. This ratio is then compared to a standard curve made at different concentrations of a known calibrant digested in the same manner as the unknown and containing the same amount of internal standard peptide. The concentration of immunoglobulins bound be a specific antigen can then be reported as an absolute concentration such as g/L.

The amount of one or more different antigen specific immunoglobulin classes, subclasses and/or light chain types in the sample may therefore be absolutely or relatively quantitated.

The immunoglobulin classes may be selected from IgG, IgA, IgM, IgD and IgE. The subclasses may be selected from IgG1, IgG2, IgG3, IgG4, IgA1 and IgA2. The light chains may be selected from lambda light chains and kappa light chains. 2, 3, 4, 5, 6, 7, 8, 9, 10, 11, 12, or 13 different classes, subclasses and types may be detected, depending on the immune response observed. IgG3, as discussed above, has a particular interest, so typically at least two classes, subclasses or types detected include IgG3. Alternatively IgE and IgG4 are most likely to be of interest to study allergies.

The relative ratio of lambda : kappa light chains may be determined

The method may additionally comprise identifying one or more of:
(a) J-chains bound to IgA and/or IgM; and/or
(b) CD5L-bound to IgM.

Detection of J-chains is described in, for example, WO 2019/055632, incorporated herein in its entirety.

Detection of CD5L (also known as CD5-like antigen and AIM - Apoptosis Inhibitor of Macrophage) is described in WO 2019/055634 incorporated herein in its entirety. The detection of J-chain in a sample provides a quick qualitative assessment of the amount of IgA and/or IgM molecules associated with immunoglobulins bound to specific antigens after elution and reduction and prior to digestion of a sample. Where such J-chains or CDL5 inhibitors are to be assayed, the serum sample may be purified or enriched for IgA or IgM, for example using anti-IgA or anti-IgM antibodies or fragments, prior to contacting the IgA or IgM-enriched sample with the antigen on the substrate.

The immunoglobulins in the sample may be purified or enriched prior to contacting the sample bound to the support. For example, IgG may be enriched in the sample using, for example, protein G or Melon gel. The immunoglobulins and sample may also be enriched using anti-immunoglobulin antibodies. For example, anti-IgM, anti-IgG, anti-IgA, anti-IgD or anti-IgE-specific antibodies, or fragments thereof. Anti-subclass specific antibodies, anti-light chain-type specific antibodies, or indeed anti-heavy chain class-light chain type specific antibodies may also be used to specifically purify immunoglobulins from the sample. Fragments of antibodies may be used.

Moreover, it may also be desirable to remove one or more types of antibody or classes of antibodies from the sample, prior to contacting with the antigen bound to the subject. For example, IgG is present in considerably higher quantities than other classes of antibodies. When studying, for example, IgA or IgM, there may be a high background of IgG. Removing IgG, for example by using protein G, Melon gel, or anti-IgG-specific antibodies or fragments thereof, may therefore be desirable. Anti-immunoglobulin class, sub-class, light chain type, heavy chain class-light chain type specific antibodies are all commercially available, for example from The Binding Site Limited, Birmingham, United Kingdom.

The antibodies or fragments thereof used to selectively remove or purify immunoglobulins from the sample may be single domain antibody fragments, as described in, for example, WO 2015/154052, incorporated herein in its entirety. Alternatively, cross-linked antibodies, in which the heavy chain is cross-linked to the light chain via means of, for example, a thioether bond or another cross-linking compound such as bis(maleimido)ethane, as disclosed in WO 2017/14490 incorporated herein in its entirety, may also be used. Use of SDAF’s and cross-linked antibodies reduces contamination by the purifying antibody of the sample with heavy or light chains, which may affect the data produced by the method of the invention.

The antigen bound to the substrate may be an antigenic portion of a larger molecule. Accordingly, for example, it may be a sub-unit or a fragment of a larger protein, which would allow the immune response to different parts of that protein to be studied.

The antigen may be an antigen from a cell, virus, a bacterium, an archaebacterium, a fungus, a protozoan, a helminth, an autoimmune antigen, a cancer antigen, an antigen capable of inducing an allergic reaction in a subject. The organisms may, for example, be pathogenic organisms against which, for example, a vaccine or one of the treatments may be being studied, or the effect of a vaccine or a treatment on the progression of a disease caused by such an organism is being followed. Furthermore, the neutralizing effect of an immunoglobulin bound to the antigen may be monitored using mass spectrometry by observing molecules associated with the killing of the antigen released into the sample buffer after binding of a neutralizing immunoglobulin.

The methods may also allow the study and identification of activating and non-activating antibodies for receptor sites.

The methods of the invention may be used to profile an antibody response prior, during or after an infection.

Examples of pathogenic viruses include: Hepatitis A, Coxsackievirus and other Picornaviridae; Hepatitis B and other Hepadaviridae; Hepatitis C, Dengue virus and other Flaviviridae; Herpes Simplex virus 1 & 2, Cytomegalovirus, Epstein Barr and other Herpesviridae; HIV and other Retroviridae; Influenza and other Orthomyxoviridae; Papillomavirus and other Papillomaviridae; Rabies and other Rhabdoviridae; Respiratory Syncytial Virus and other Paramyxoviridae; SARS Cov 2 and MERS and other Coronaviridae.

Examples of pathogenic bacteria include: Staphylococcus (e.g. S. aureus), Streptococcus, Escherichia coli, Neisseria, Pseudomonas, Mycobacterium tuberculosis, Yersinia, Bacillus, Clostridium (Clostridium difficile and Clostridium botulinum), Haemophilus (Haemophilus influenza), Listeria, Borrelia and Rickettsia.

Pathogenic fungi include: Coccidiodes immitis, Histoplasma capsulatum, Bastomyces and Pneumocystis

The antigen may be from a protozoan, such as malaria or a trypanosome. Common infectious diseases caused by protozoans include malaria, giardia and toxoplasmosis. Additionally, dysentery, may be caused by a number of amoeba. These include Entamoeba histolytica, Trypanosoma brucei gambiense, Leishmania donovani, Plasmodium vivax, Plasmodium malariae, Plasmodium falciparumand Toxoplasma gondil. Such protozoan diseases are often difficult to produce vaccines to as the organisms often have systems that evade the immune system. Being able to study immune response in a subject to such a disease is likely to help characterise the disease and hopefully identify possible vaccine components.

There are a number of helminth diseases or organisms, such as tapeworms, flukes, ascariasis, trichurias, hookworm, enterobiasis, strongyloidiasis, filariasis, trichinosis, dirofilariasis and angiostrongyliasis (rat lungworm disease).

Autoimmune diseases are often very debilitating. These include, for example, rheumatoid arthritis, systemic lupus erythematosus (SLE), mixed connective tissue disease (MCTD), inflammatory bowel disease, multiple sclerosis, type 1 diabetes mellitus, Guillain-Barre, chronic inflammatory demyelinating polyneuropathy and psoriasis. The ability to characterise the diseases and identify antigens which induce the diseases, or to which antibodies or subclasses of antibodies bind, will assist in the identification of treatment strategies and indeed characterisation of the disease and progression of the diseases in subjects. The antigen may also be a cancer antigen.

A number of cancers express cancer antigens, such as MHC class I or class II molecules, on the surface of tumours or otherwise secrete them into the body. Neoantigens are also being identified for cancers. Again, the method of the invention will allow the characterisation and detection of such cancers and assist in identification of novel treatments.

Subjects may become sensitised to antigens, for example to pollen, bee stings or, for example, nickel. Different symptoms are observed when having such an allergy, be they asthma, skin itching, or more extreme reactions such as anaphylactic shock, often dependent on different types of antibodies or the amounts of such different types of antibodies within the system of the subject. IgE and IgG4 are most likely to be of interest for such conditions.

The antigen may be a virus antigen, for example, a viral envelope protein, a capsid protein, an enzyme or haemagglutinin. Whole viral lysates or complex extracts could also be utilised. Enzymes include, for example, neuraminidase or methyltransferase. The latter being often found in coronaviruses, such as SARS-CoV-2. Matrix proteins are often known as “M1” proteins. Ion channel proteins are found in some viruses, and are also known as “M2” proteins.

Similarly, the bacterial antigen may be at least one antigenic portion of a cellular antigen, a flagella antigen, a somatic antigen, a virulent antigen, a fimbrial antigen or a toxoid.

The subject may be any antibody-producing organism, such as a fish, a mammal, a bird or a reptile. More typically, the subject is a mammal, such as a human, a non-human ape, a monkey, a horse, a sheep, a goat, a cow, a dog, a cat, or a rodent such as a mouse, hamster or rat. The mammal may also be a camelid. The latter are of particular interest because they typically produce antibodies lacking a light chain and are becoming increasingly used to produce, for example, SDAF (single domain antibody fragments).

The sample is typically any biological fluid, such as blood, serum, plasma, cerebrospinal fluid, urine, tears, sputum, lavage fluid or saliva. The lavage fluid may, for example, be bronchoalveolar lavage fluid which may be obtained, for example, via a bronchoscopy. Nasopharyngeal or oropharyngeal swab samples, as additionally nasal secretions, may also be used.

An ionisation control may be added to the sample, prior to carrying out the mass spectrometry. Such ionisation controls are typically proteins of a different molecular mass to the compounds to be identified by the mass spectrometry. This ensures that the mass spectrometry technique is operating consistently between samples.

The methods of the invention allows the production of a matrix of, for example, different antigens against different immune responses as measured by the different antigen-specific immunoglobulin classes, subclasses, or light chain types identified by the method of the invention. This allows variations in the immune response against different antigens to be readily identified. Accordingly, a further aspect of the invention provides a method of producing a matrix or profile.

A method of the invention may be combined with one or more additional indicators of the immune response or immune function in a subject. These include immunoregulatory and proinflammatory cytokines, for example, interferons, interleukins, interleukin-1, interleukin-2, TNF-Alpha, the numbers of circulating macrophages or other white blood cells within the blood or other fluid, such as lavage fluid, the amounts of complement proteins in a sample and other such factors.

The instrumentation used for analysis may for example consist of a liquid chromatograph coupled with a mass spectrometer (LC-MS, LC-MS/MS). Other instrument configurations include, but are not limited to, CZE coupled with a mass spectrometer or an ion mobility device coupled with a mass spectrometer. Typical ionization techniques used include but are not limited to electrospray ionisation and MALDI ionization. A mass spectrometer used for analysis may include but is not limited to, a quadropole time-of-flight mass spectrometer an orbitrap mass spectrometer, a triple quadrupole mass spectrometer, an ion trap mass spectrometer or a time-of-flight mass spectrometer.

Methods of selecting one or more vaccine targets comprising the use of the method of the invention is provided as are methods of identifying an immune status of subject targets comprising the use of a method of the invention. Methods of characterising an immune response in the subject to a pathogen, allergen or other antigen comprising the use of a method of the invention is still further provided. The severity or progression or treatment of a condition caused by a pathogen may be determined. A still further aspect of the invention provides a method of characterising an autoimmune response in the subject comprising a use of the method of the invention, or characterising an allergic response in a subject comprising a use or the method of the invention. The method may also be used to monitor the progression of a disease in the subject.

The method of the invention may also provide evidence of the optimum types of antibody or subclass of antibody to use or to stimulate the maximum response to an antigen. For example, this may allow a monoclonal antibody class or other monoclonal antibody characteristics to be optimised.

A still further aspect of the invention provides a computer implemented method for identifying or characterising an immune response in a subject, comprising the use of the method of the invention. The method may include comparing a mass spectrum obtained for a first antigen specific immunoglobulin class, subclass and/or light chain type with a mass spectrum for a second antigen specific immunoglobulin class, substrate class and/or light chain type, wherein the mass spectrum is obtained by a method of the invention. The mass spectrum may be received by the computer and compared, for example to provide an amount or ratio of one or more peaks associated with the class, subclass or light chain type

The computer may comprise a computer processor and a computer memory.

Apparatus for identifying or characterising an immune response in a subject by a method according to the invention and comprising a use of a computer implementing method according to the invention is also provided. The apparatus may comprise a mass spectrometer.

Assay kits for use in the method of the invention comprising a plurality of antigens attached to one or more substrates in combination with one or more immunoglobulin calibrators is also provided.

The invention will now be described by way of example only with reference to the following figures:

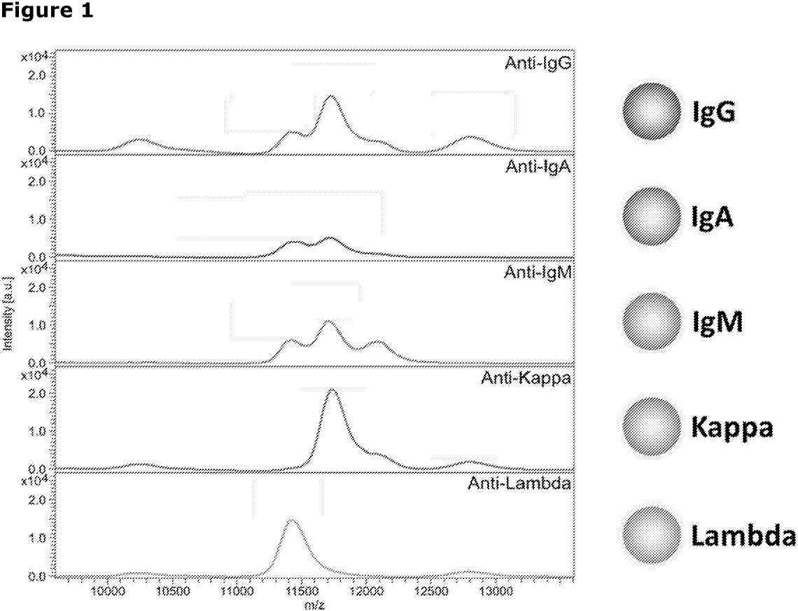

FIG. 1 shows five mass spectra for immunoglobulins which have been immunopurified with anti-IgG, anti-IgA, anti-IgM, anti-kappa and anti-lambda antibodies. After immunopurification, the heavy chains and light chains were dissociated using a reducing agent, dithiothreitol, prior to performing mass spectrometry using MALDI-TOF.

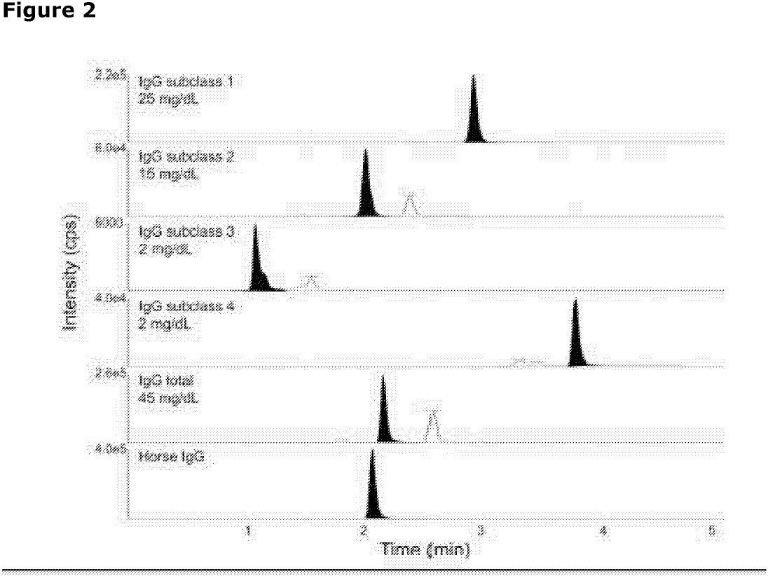

FIG. 2 is from Ladwig, P.M. etal., Clinical Chemistry (2014), Volume 16, pages 1080-1088. It shows that an LC-MS/MS ion chromatogram of an IgG sub-low control (The Binding Site Limited, Birmingham, United Kingdom) diluted in 1:16 bovine serum showing a peak (shaded) used with quantification of each individual subclass peptide, the common peptide used for IgG total, and the horse IgG peptide used for quantification. A number of non-specific background peaks seen were chromatographically separated and did not interfere.

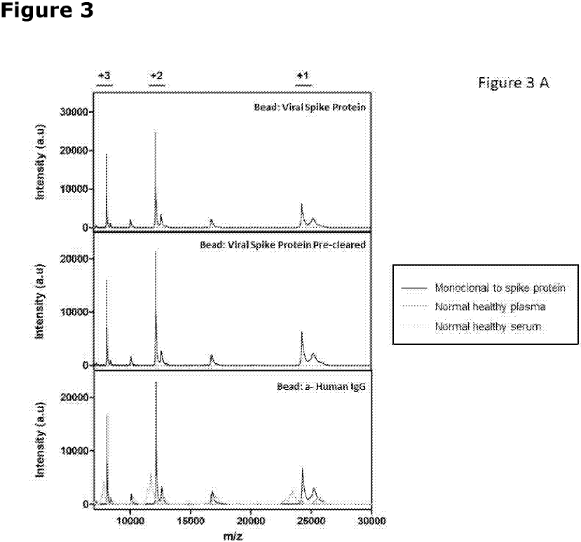

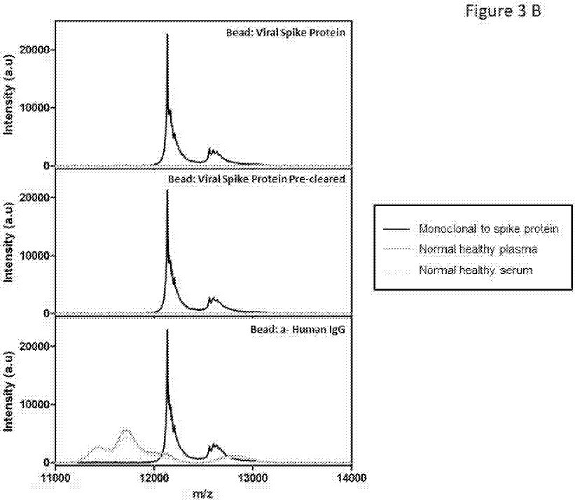

FIG. 3 shows MALDI-TOF spectra using a Coronavirus viral spike protein immobilised in a paramagnetic bead. The spectra are presented over an extended 7000-30000 m/z range (showing all 3 charge states, A) and reduced 11000 to 14000 m/z range (swing only the +2-charge state, B). A monoclonal antibody (specific to the spike protein) was bound by the bead, eluted, and peak separated via MALDI-TOF mass spectrometry. Additionally, healthy human plasma and serum were also separately incubated with the viral spike protein bead. The bead marked viral spike protein (pre-cleared) was a control to demonstrate that post-conjugation bead washing did not damage the immobilised protein. The bead marked α-human IgG is a bead in which the viral protein has been replaced by an α-IgG-specific antibody to demonstrate that that antibody binds not only the monoclonal antibody, which is an IgG monoclonal antibody, but when used with normal plasma and normal serum, also binds to the immunoglobulins in the normal plasma and normal serum.

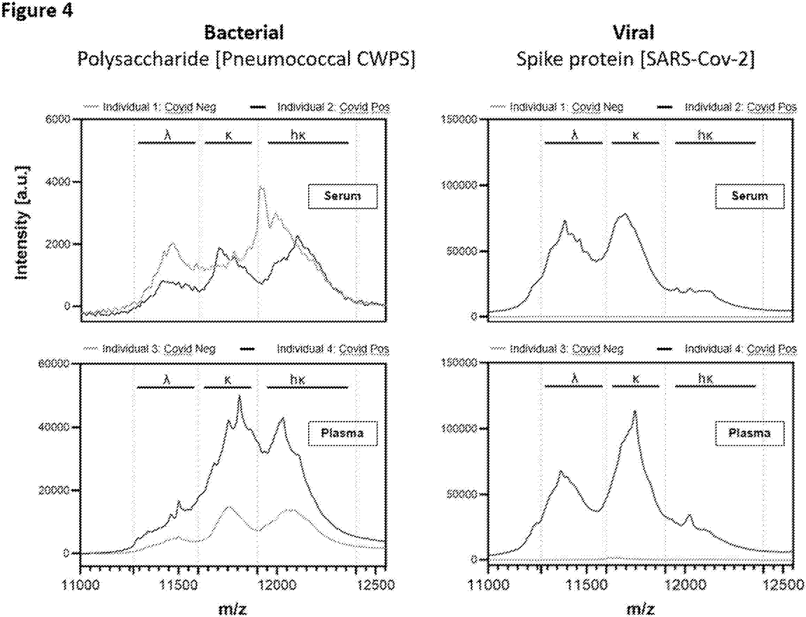

FIG. 4 MALDI mass spectrometry spectra from Covid-19 negative (healthy) and PCR-positive (diseased) individuals tested against viral SARS-CoV-2 Spike protein and bacterial Pneumococcal cell wall polysaccharide (CWPS) . Serum or plasma samples of 4 individuals were immunocaptured with antigen conjugated beads Individuals 2 and 4 were “diseased” (tested positive for Covid-19). Individuals 1 and 3 were classes “healthy” (tested negative for Covid-19).

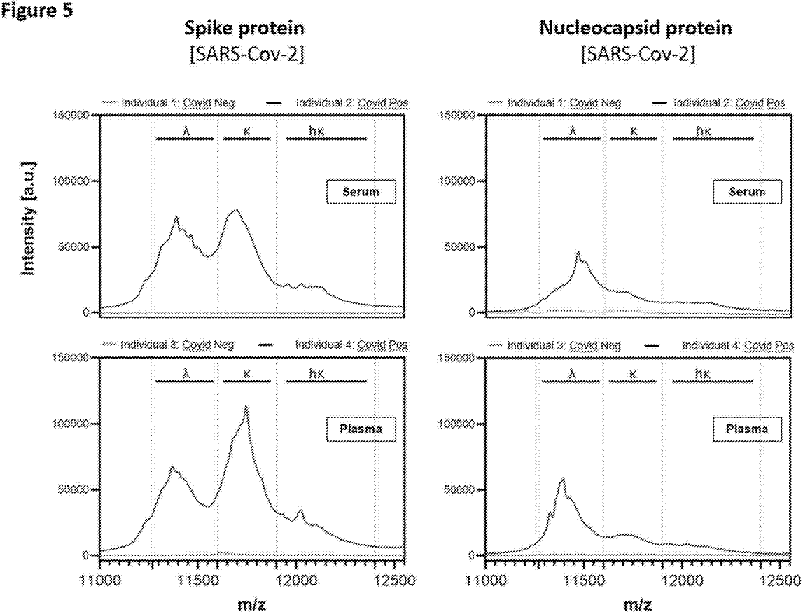

FIG. 5 MALDI mass spectrometry spectra from healthy and diseased individuals tested against a targeted infection using various infection specific antigens. SARS-CoV-2 Virus specific antigens were conjugated to beads with the viral Spike protein and Nucleocapsid protein. Samples of 4 individuals were immunocaptured with antigen conjugated beads. Two of the individuals were “diseased” (tested positive for Covid-19, individual 2 and 4) and two healthy (individuals 1 and 3).

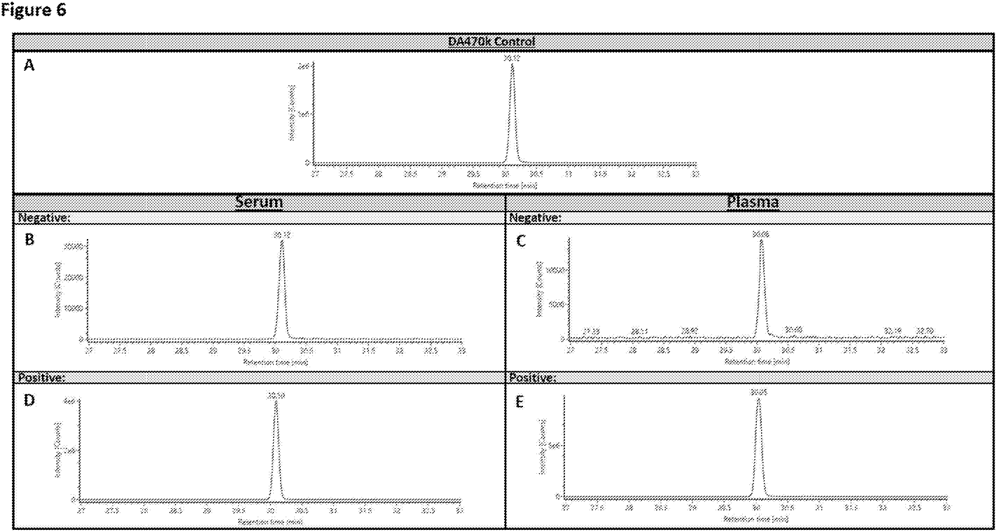

FIG. 6 Extracted ion chromatograms (XIC) of IgG1 peptide TPEVTC(CAM)VVVDVSHEDPEVK detected within the digest of ERM-DA470k reference serum and eluate digests of serum and plasma samples immunoprecipitated using beads conjugated with SARS-CoV-2 spike protein. (A) XIC of IgG1 peptide in ERM-DA470k. (B-E) XICs of IgG1 peptide in eluate digests of immunoprecipitated (B) COVID-19 negative serum, (C) COVID-19 negative plasma, (D) COVID-19 positive serum, and (E) COVID-19 positive plasma. *(CAM) = carbamidomethylated cysteine.

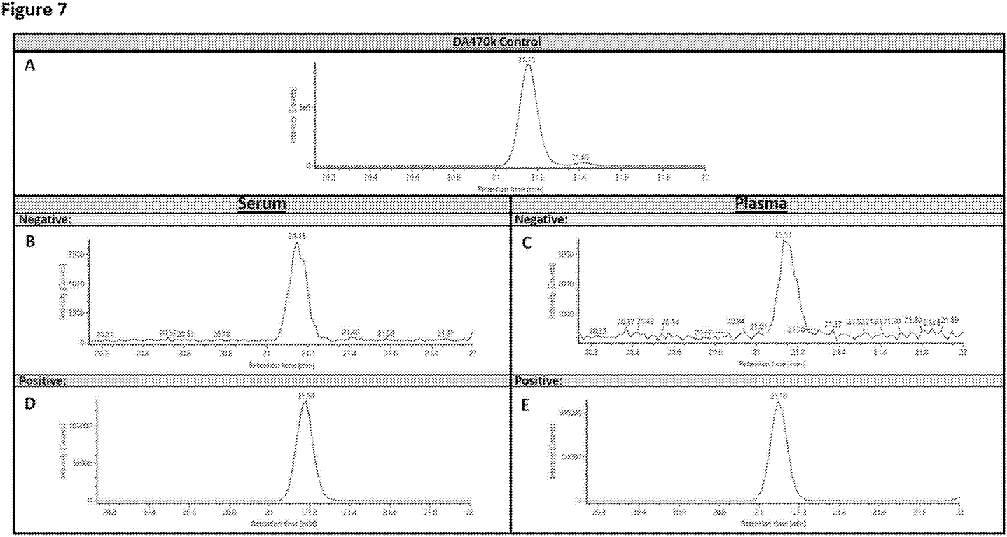

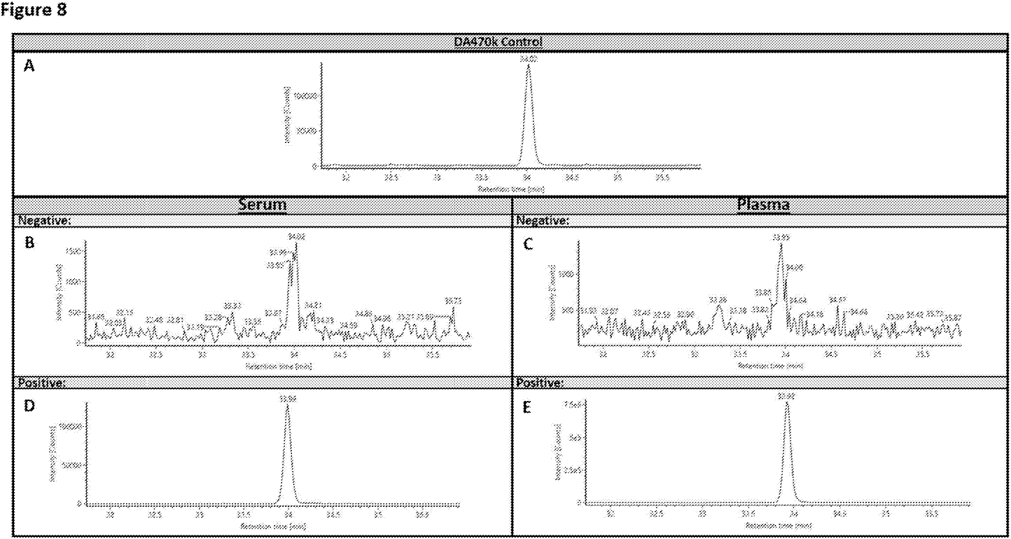

FIG. 7 Extracted ion chromatograms (XIC) of IgG2 peptide GLPAPIEK detected within the digest of ERM-DA470k reference serum and eluate digests of serum and plasma samples immunoprecipitated using beads conjugated with SARS-CoV-2 spike protein. (A) XIC of IgG2 peptide in ERM-DA470k. (B-E) XICs of IgG2 peptide in eluate digests of immunoprecipitated (B) COVID-19 negative serum, (C) COVID-19 negative plasma, (D) COVID-19 positive serum, and (E) COVID-19 positive plasma.

FIG. 8 Extracted ion chromatograms (XIC) of IgG3 peptide TPEVTC(CAM)VVVDVSHEDPEVQFK detected within the digest of ERM-DA470k reference serum and eluate digests of serum and plasma samples immunoprecipitated using beads conjugated with SARS-CoV-2 spike protein. (A) XIC of IgG3 peptide in ERM-DA470k. (B-E) XICs of IgG3 peptide in eluate digests of immunoprecipitated (B) COVID-19 negative serum, (C) COVID-19 negative plasma, (D) COVID-19 positive serum, and (E) COVID-19 positive plasma. *(CAM) = carbamidomethylated cysteine.

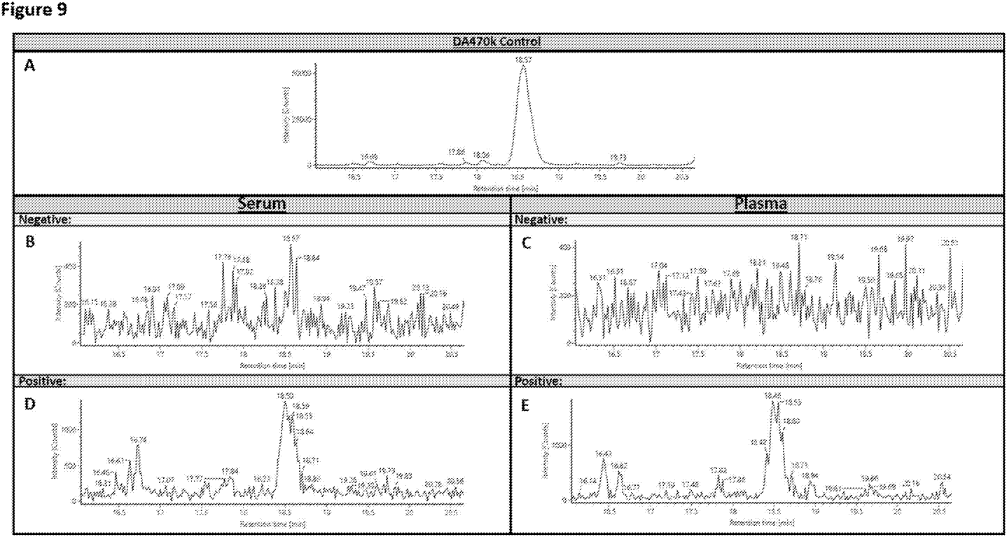

FIG. 9 Extracted ion chromatograms (XIC) of IgG4 peptide GLPSSIEK detected within the digest of ERM-DA470k reference serum and eluate digests of serum and plasma samples immunoprecipitated using beads conjugated with SARS-CoV-2 spike protein. (A) XIC of IgG4 peptide in ERM-DA470k. (B-E) XICs of IgG4 peptide in eluate digests of immunoprecipitated (B) COVID-19 negative serum, (C) COVID-19 negative plasma, (D) COVID-19 positive serum, and (E) COVID-19 positive plasma.

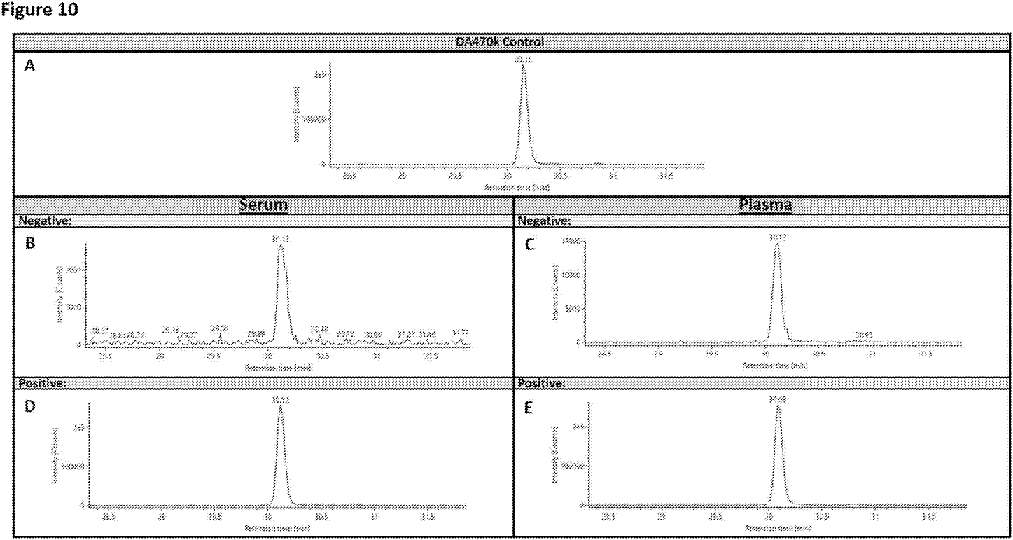

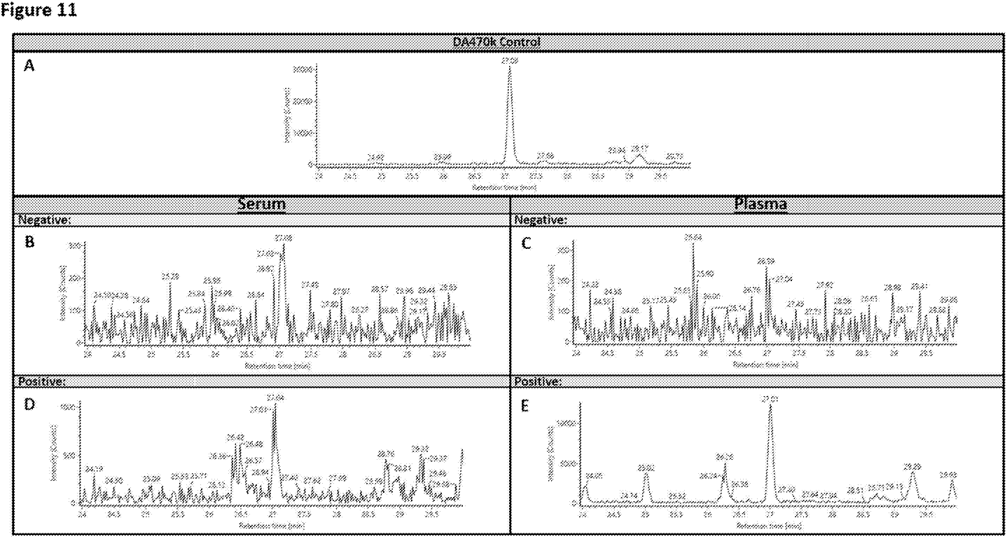

FIG. 10 Extracted ion chromatograms (XIC) of IgA1 peptide DASGVTFTWTPSSGK detected within the digest of ERM-DA470k reference serum and eluate digests of serum and plasma samples immunoprecipitated using beads conjugated with SARS-CoV-2 spike protein. (A) XIC of IgA1 peptide in ERM-DA470k. (B-E) XICs of IgA1 peptide in eluate digests of immunoprecipitated (B) COVID-19 negative serum, (C) COVID-19 negative plasma, (D) COVID-19 positive serum, and (E) COVID-19 positive plasma.

FIG. 11 Extracted ion chromatograms (XIC) of IgA2 peptide DASGATFTWTPSSGK detected within the digest of ERM-DA470k reference serum and eluate digests of serum and plasma samples immunoprecipitated using beads conjugated with SARS-CoV-2 spike protein. (A) XIC of IgA2 peptide in ERM-DA470k. (B-E) XICs of IgA2 peptide in eluate digests of immunoprecipitated (B) COVID-19 negative serum, (C) COVID-19 negative plasma, (D) COVID-19 positive serum, and (E) COVID-19 positive plasma.

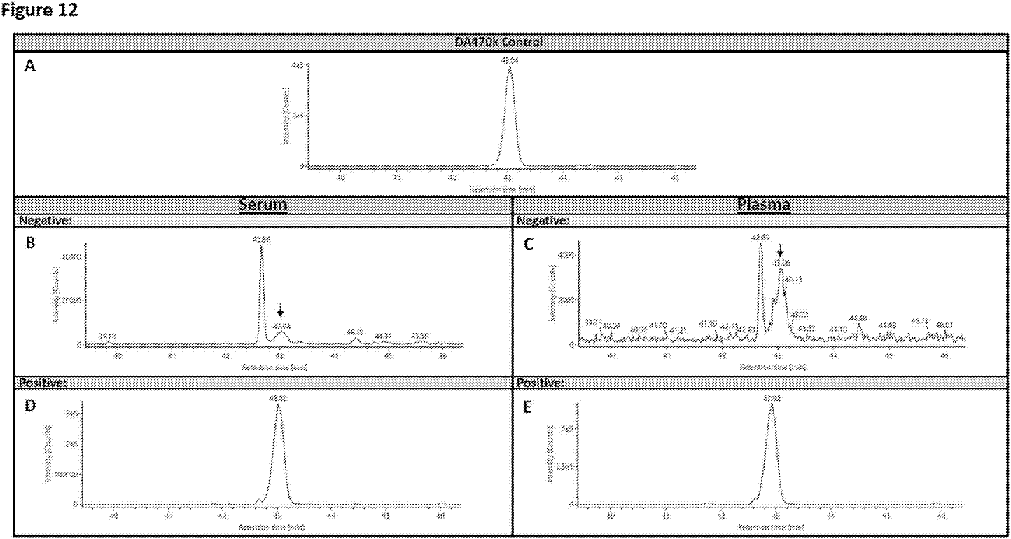

FIG. 12 Extracted ion chromatograms (XIC) of kappa LC peptide SGTASVVC(CAM)LLNNFYPR detected within the digest of ERM-DA470k reference serum and eluate digests of serum and plasma samples immunoprecipitated using beads conjugated with SARS-CoV-2 spike protein. (A) XIC of kappa LC peptide in ERM-DA470k. (B-E) XICs of kappa LC peptide in eluate digests of immunoprecipitated (B) COVID-19 negative serum, (C) COVID-19 negative plasma, (D) COVID-19 positive serum, and (E) COVID-19 positive plasma. *(CAM) = carbamidomethylated cysteine.

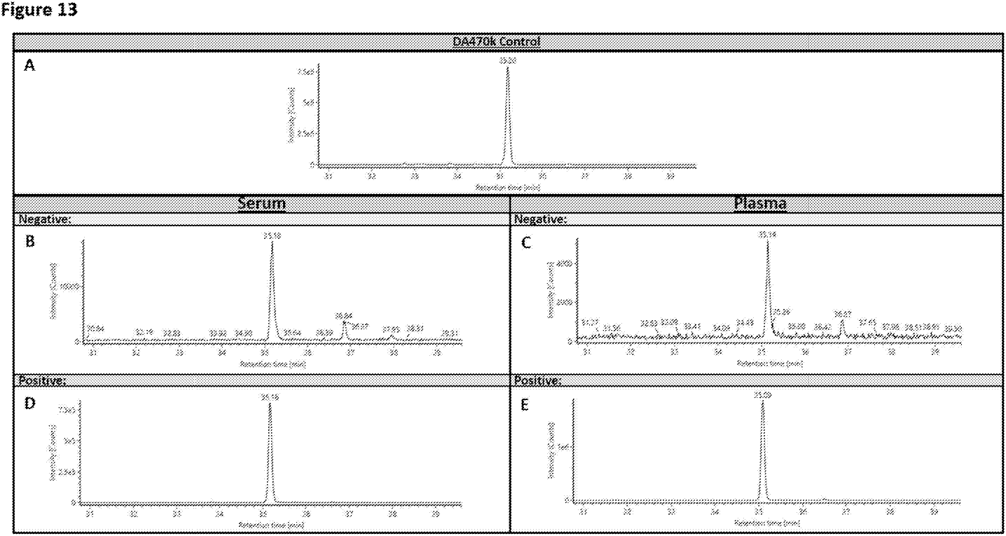

FIG. 13 Extracted ion chromatograms (XIC) of lambda LC peptide YAASSYLSLTPEQWK detected within the digest of ERM-DA470k reference serum and eluate digests of serum and plasma samples immunoprecipitated using beads conjugated with SARS-CoV-2 spike protein. (A) XIC of lambda LC peptide in ERM-DA470k. (B-E) XICs of lambda LC peptide in eluate digests of immunoprecipitated (B) COVID-19 negative serum, (C) COVID-19 negative plasma, (D) COVID-19 positive serum, and (E) COVID-19 positive plasma.

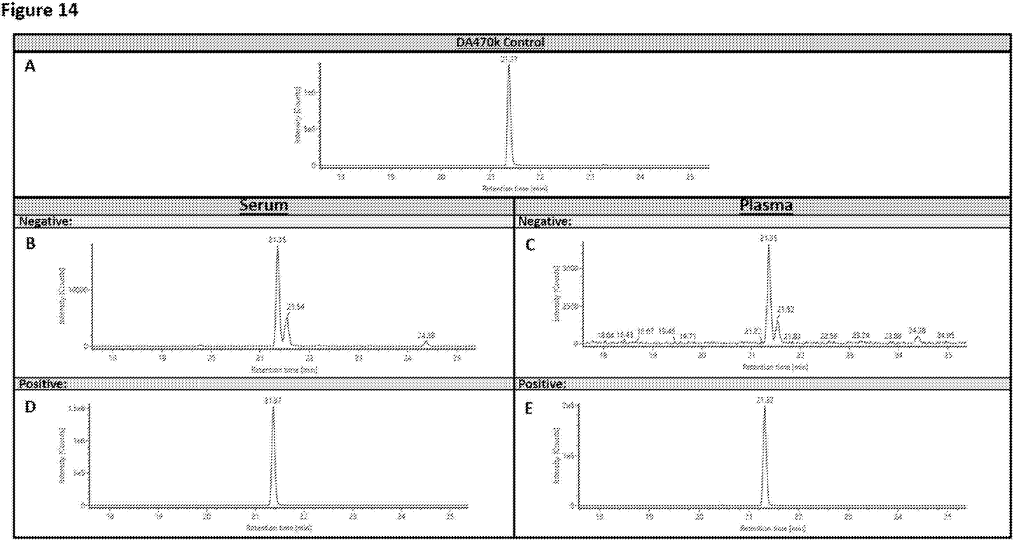

FIG. 14 Extracted ion chromatograms (XIC) of IgG peptide DTLMISR detected within the digest of ERM-DA470k reference serum and eluate digests of serum and plasma samples immunoprecipitated using beads conjugated with SARS-CoV-2 spike protein. (A) XIC of IgG peptide in ERM-DA470k. (B-E) XICs of IgG peptide in eluate digests of immunoprecipitated (B) COVID-19 negative serum, (C) COVID-19 negative plasma, (D) COVID-19 positive serum, and (E) COVID-19 positive plasma.

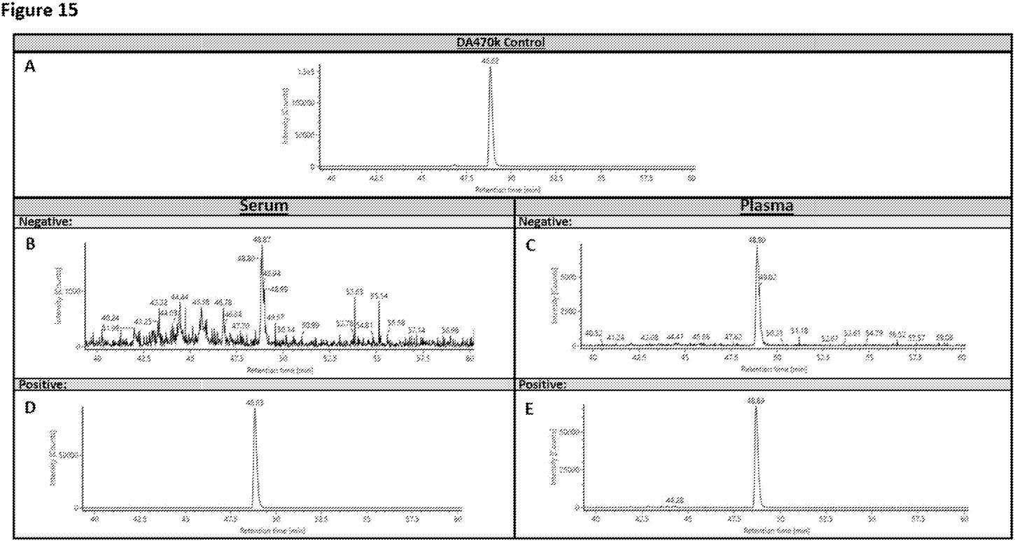

FIG. 15 Extracted ion chromatograms (XIC) of IgA peptide SGNTFRPEVHLLPPPSEELALNELVTLTC(CAM)LAR detected within the digest of ERM-DA470k reference serum and eluate digests of serum and plasma samples immunoprecipitated using beads conjugated with SARS-CoV-2 spike protein. (A) XIC of IgA peptide in ERM-DA470k. (B-E) XICs of IgA peptide in eluate digests of immunoprecipitated (B) COVID-19 negative serum, (C) COVID-19 negative plasma, (D) COVID-19 positive serum, and (E) COVID-19 positive plasma. *(CAM) = carbamidomethylated cysteine.

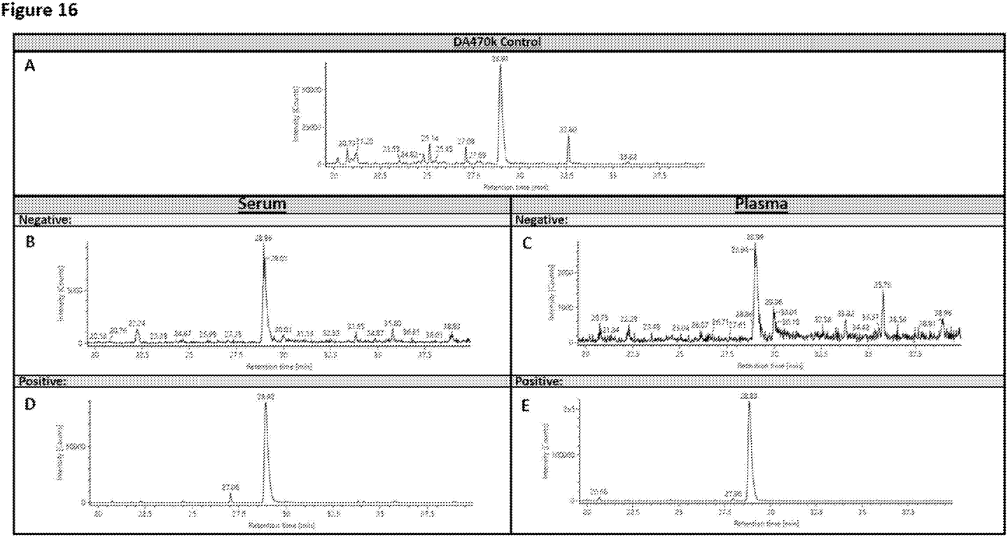

FIG. 16 Extracted ion chromatograms (XIC) of IgM peptide GVALHRPDVYLLPPAR detected within the digest of ERM-DA470k reference serum and eluate digests of serum and plasma samples immunoprecipitated using beads conjugated with SARS-CoV-2 spike protein. (A) XIC of IgM peptide in ERM-DA470k. (BE) XICs of IgM peptide in eluate digests of immunoprecipitated (B) COVID-19 negative serum, (C) COVID-19 negative plasma, (D) COVID-19 positive serum, and (E) COVID-19 positive plasma.

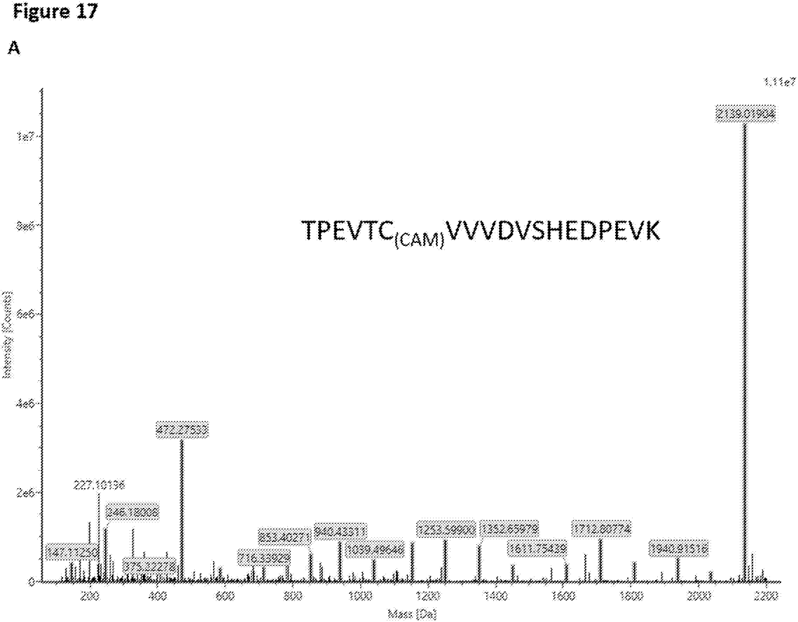

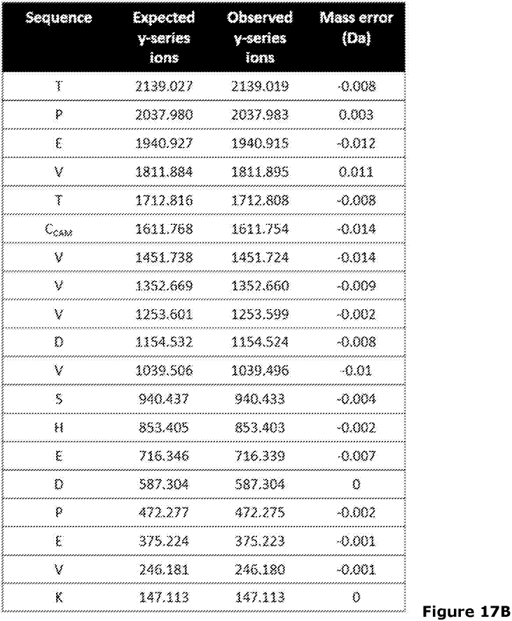

FIG. 17 Fragmentation spectrum corresponding to IgG1 peptide TPEVTC(CAM)VVVDVSHEDPEVK within digested eluate of COVID-19 positive serum immunoprecipitated with beads conjugated with SARS-CoV-2 spike protein. (A) Fragmentation spectrum highlighting the C-terminal fragment ions (y-ion series). (B) Table of expected and observed y-ion series fragments. *(CAM)= carbamidomethylated cysteine.

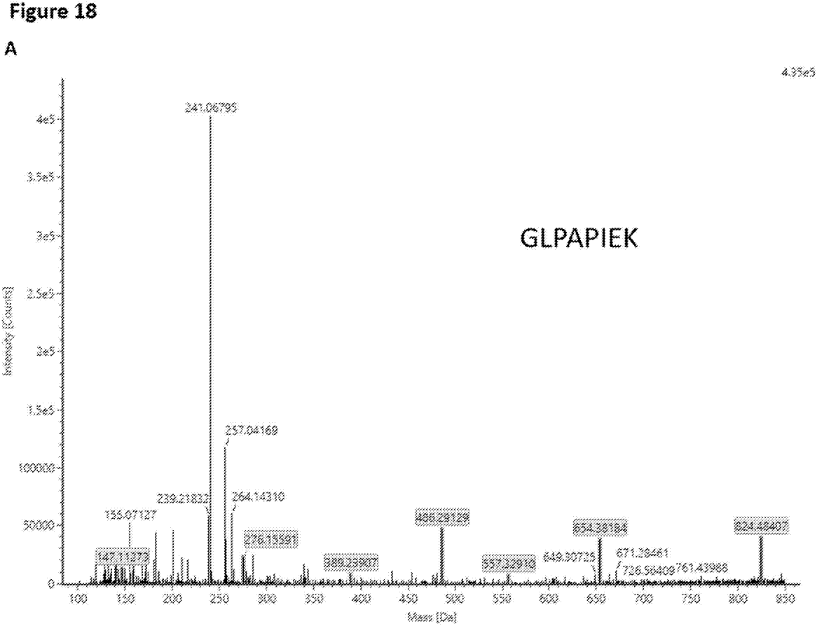

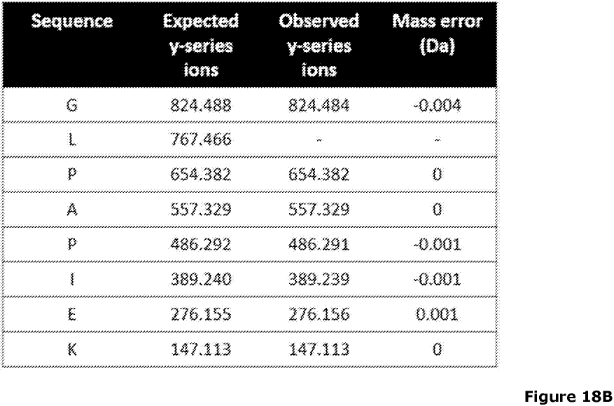

FIG. 18 Fragmentation spectrum corresponding to IgG2 peptide GLPAPIEK within digested eluate of COVID-19 positive serum immunoprecipitated with beads conjugated with SARS-CoV-2 spike protein. (A) Fragmentation spectrum highlighting the C-terminal fragment ions (y-ion series). (B) Table of expected and observed y-ion series fragments.

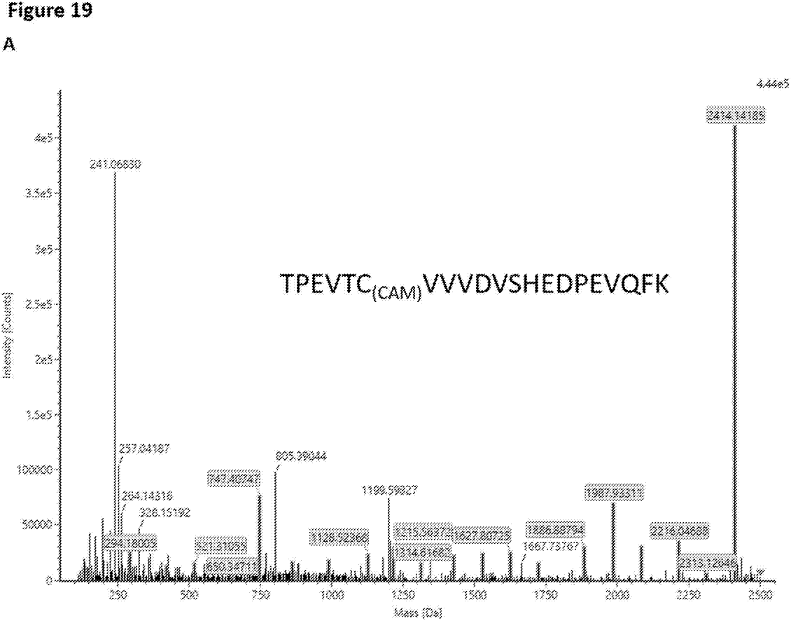

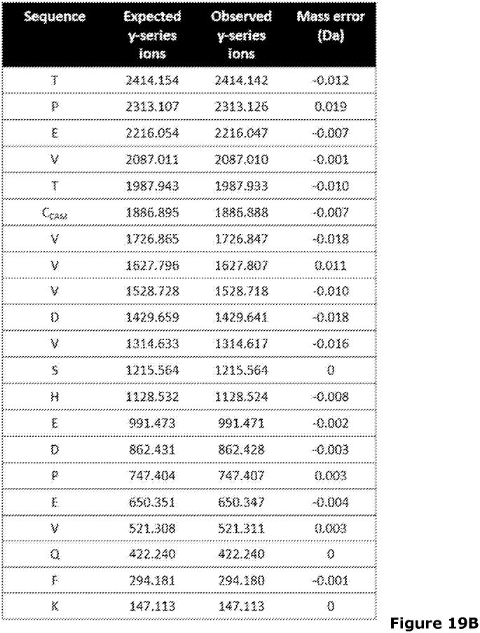

FIG. 19 Fragmentation spectrum corresponding to IgG3 peptide TPEVTC(CAM)VVVDVSHEDPEVQFK within digested eluate of COVID-19 positive serum immunoprecipitated with beads conjugated with SARS-CoV-2 spike protein. (A) Fragmentation spectrum highlighting the C-terminal fragment ions (y-ion series). (B) Table of expected and observed y-ion series fragments. *(CAM)= carbamidomethylated cysteine.

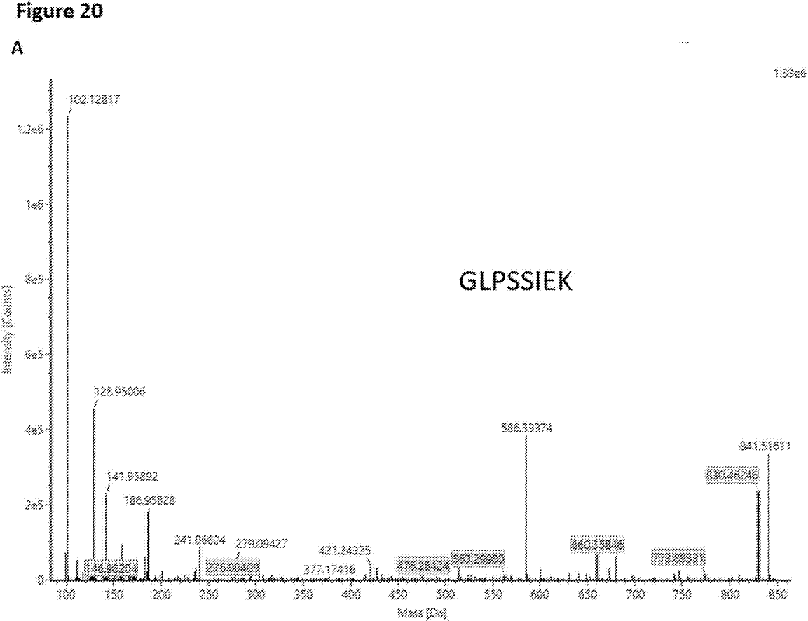

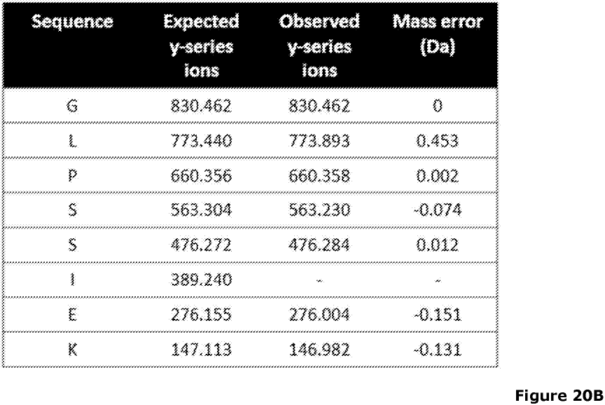

FIG. 20 Fragmentation spectrum corresponding to IgG4 peptide GLPSSIEK within digested ERM-DA470k reference serum. (A) Fragmentation spectrum highlighting the C-terminal fragment ions (y-ion series). (B) Table of expected and observed y-ion series fragments.

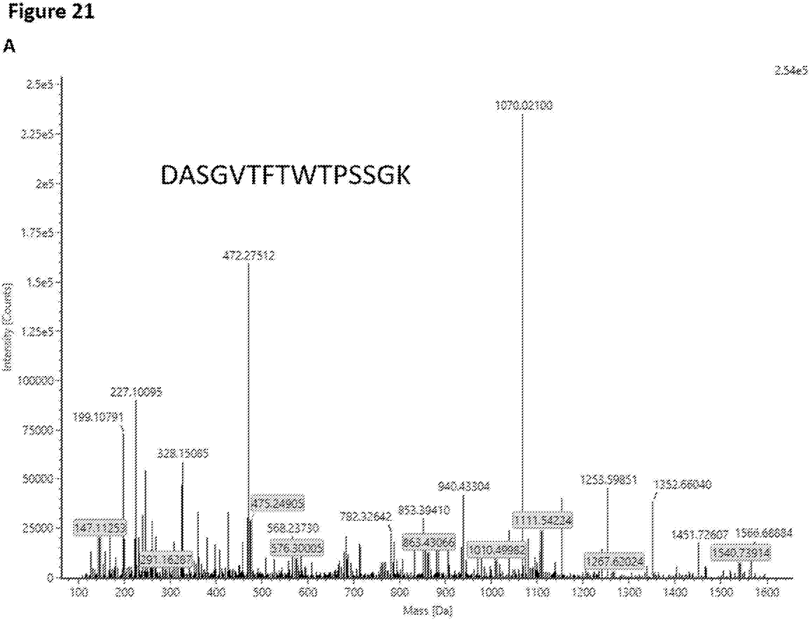

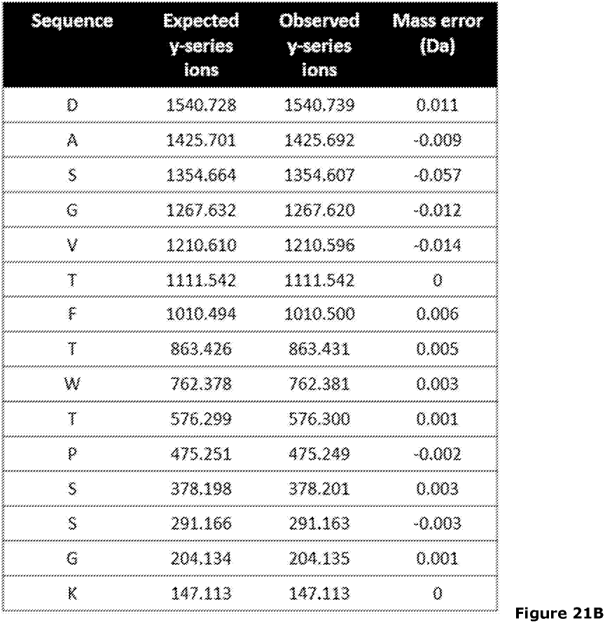

FIG. 21 Fragmentation spectrum corresponding to IgA1 peptide DASGVTFTWTPSSGK within digested eluate of COVID-19 positive serum immunoprecipitated with beads conjugated with SARS-CoV-2 spike protein. ((A) Fragmentation spectrum highlighting the C-terminal fragment ions (y-ion series). (B) Table of expected and observed y-ion series fragments.

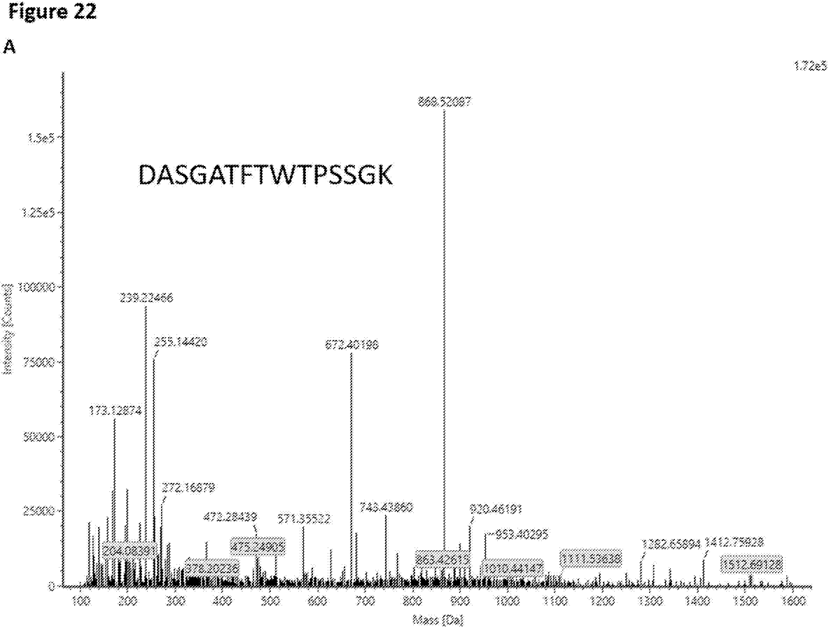

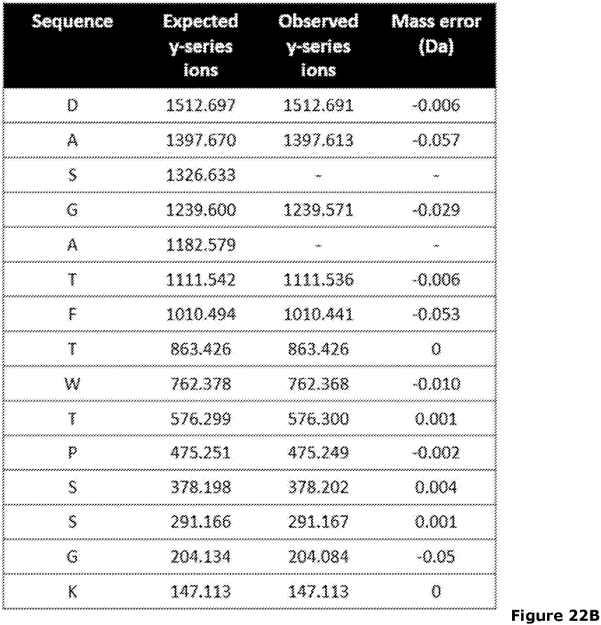

FIG. 22 Fragmentation spectrum corresponding to IgA2 peptide DASGATFTWTPSSGK within digested eluate of COVID-19 negative serum immunoprecipitated with beads conjugated with SARS-CoV-2 nucleocapsid protein. (A) Fragmentation spectrum highlighting the C-terminal fragment ions (y-ion series). (B) Table of expected and observed y-ion series fragments.

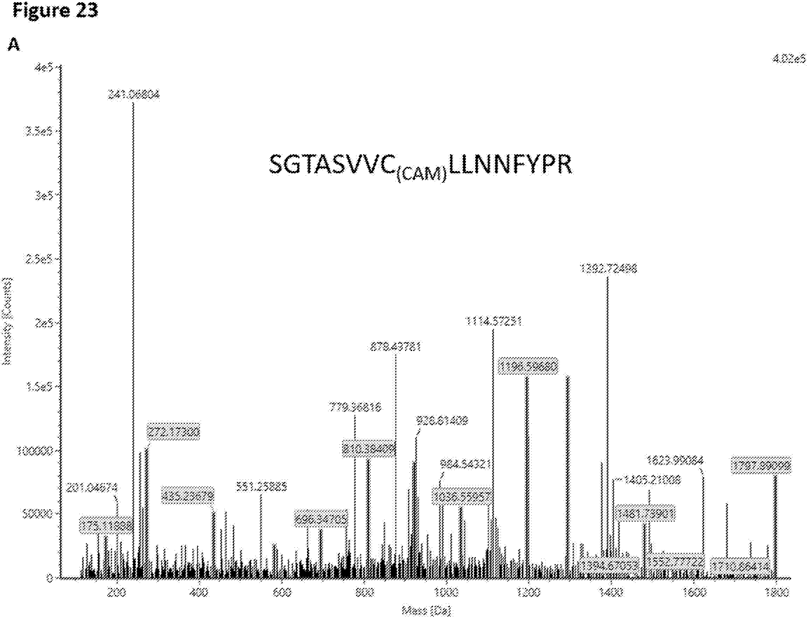

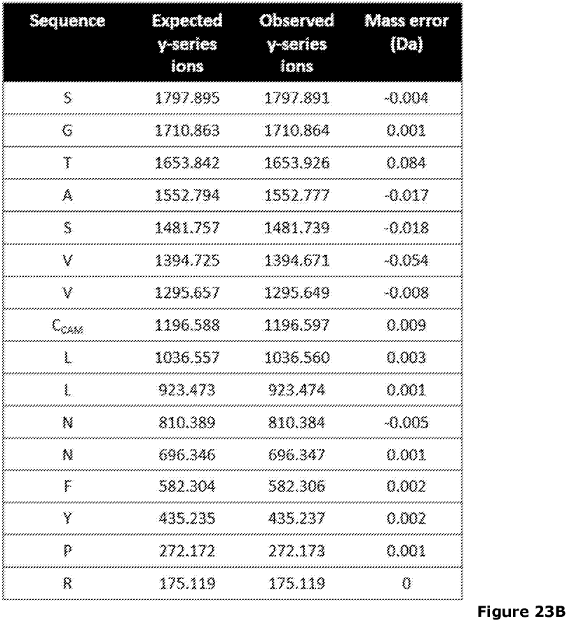

FIG. 23 Fragmentation spectrum corresponding to kappa LC peptide SGTASVVC(CAM)LLNNFYPR within digested eluate of COVID-19 positive serum immunoprecipitated with beads conjugated with SARS-CoV-2 spike protein. (A) Fragmentation spectrum highlighting the C-terminal fragment ions (y-ion series). (B) Table of expected and observed y-ion series fragments. *(CAM)= carbamidomethylated cysteine.

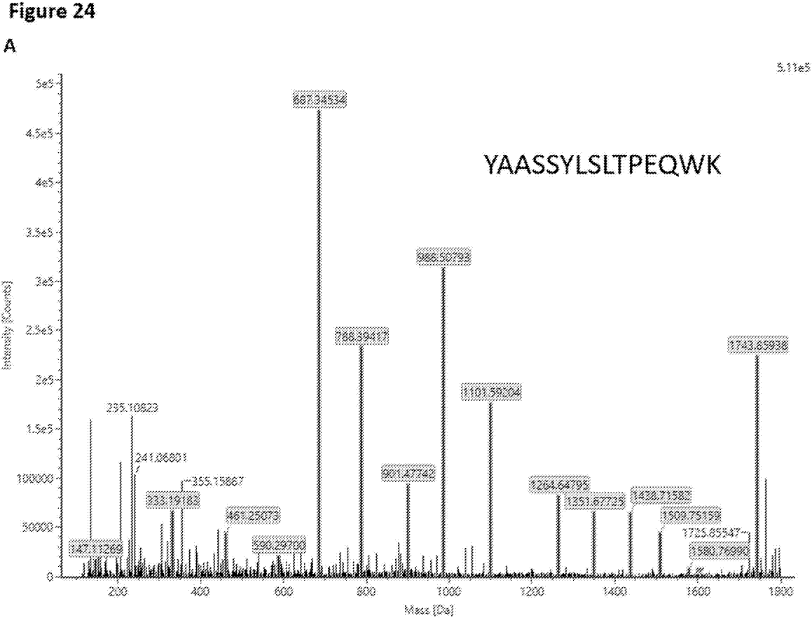

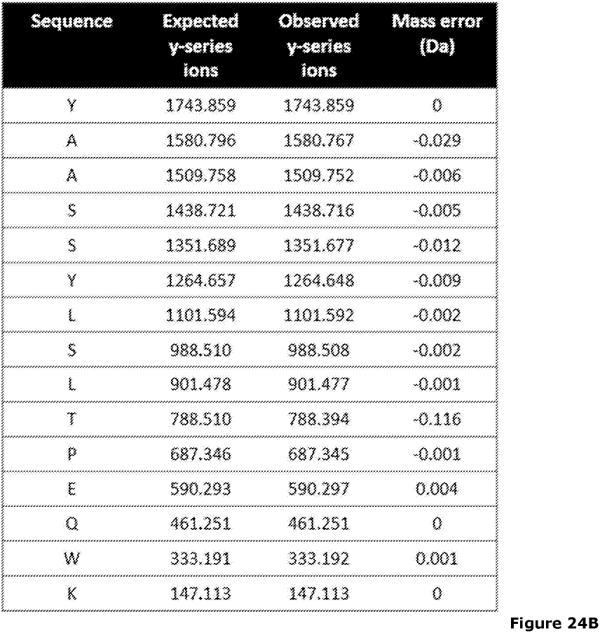

FIG. 24 Fragmentation spectrum corresponding to lambda LC peptide YAASSYLSLTPEQWK within digested eluate of COVID-19 positive serum immunoprecipitated with beads conjugated with SARS-CoV-2 spike protein (A) Fragmentation spectrum highlighting the C-terminal fragment ions (y-ion series). (B) Table of expected and observed y-ion series fragments.

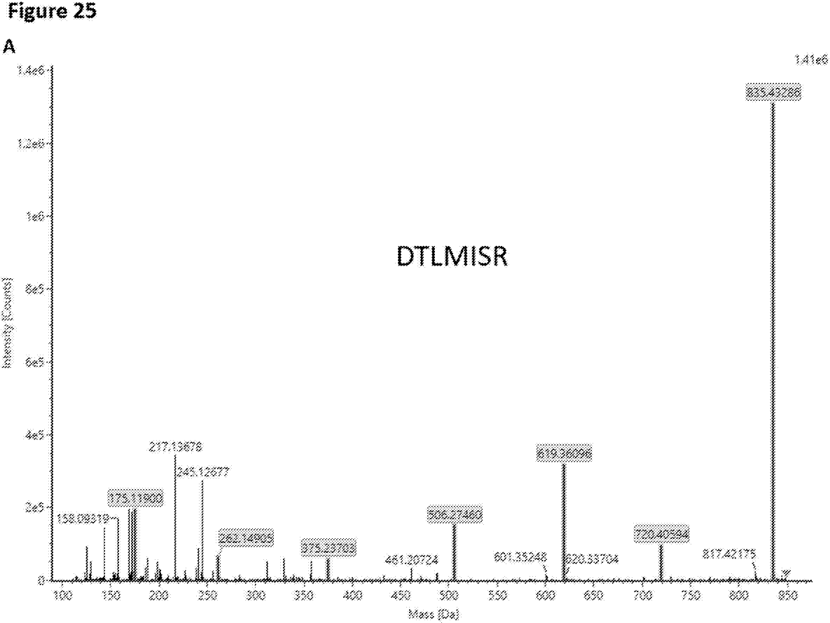

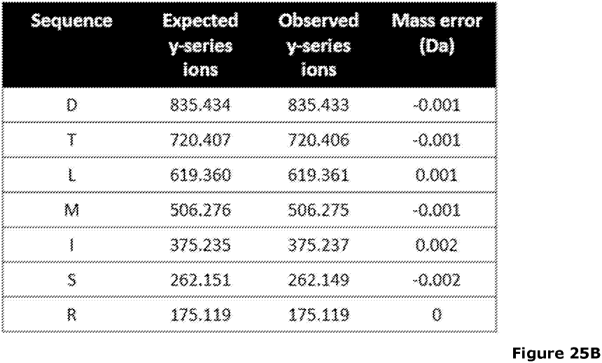

FIG. 25 Fragmentation spectrum corresponding to IgG peptide DTLMISR within digested eluate of COVID-19 positive serum immunoprecipitated with beads conjugated with SARS-CoV-2 spike protein. (A) Fragmentation spectrum highlighting the C-terminal fragment ions (y-ion series). (B) Table of expected and observed y-ion series fragments.

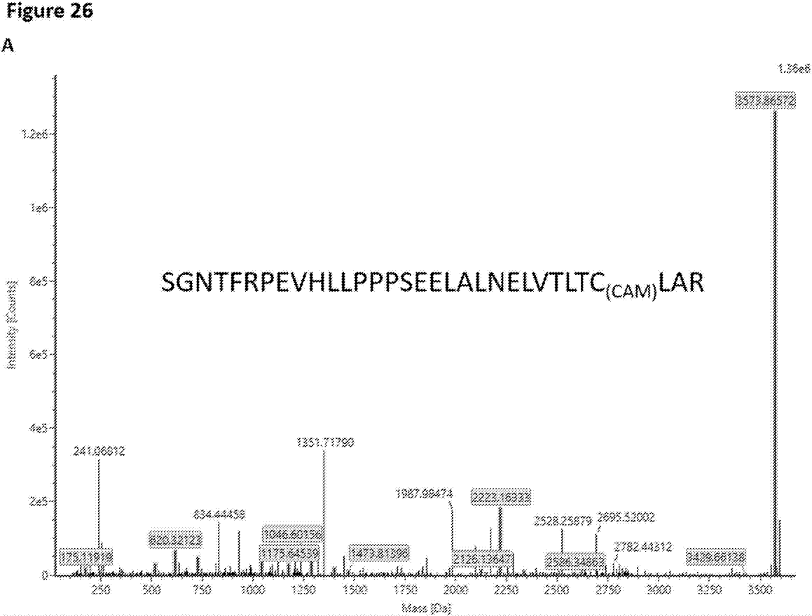

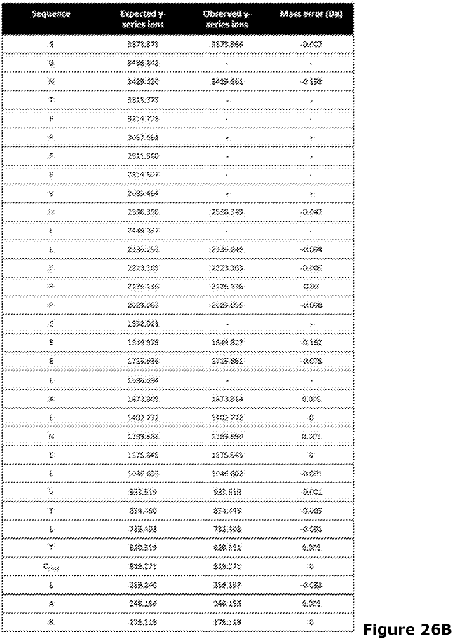

FIG. 26 Fragmentation spectrum corresponding to IgA peptide SGNTFRPEVHLLPPPSEELALNELVTLTC(CAM)LAR within digested eluate of COVID-19 positive serum immunoprecipitated with beads conjugated with SARS-CoV-2 spike protein. (A) Fragmentation spectrum highlighting the C-terminal fragment ions (y-ion series). (B) Table of expected and observed y-ion series fragments. *(CAM) = carbamidomethylated cysteine.

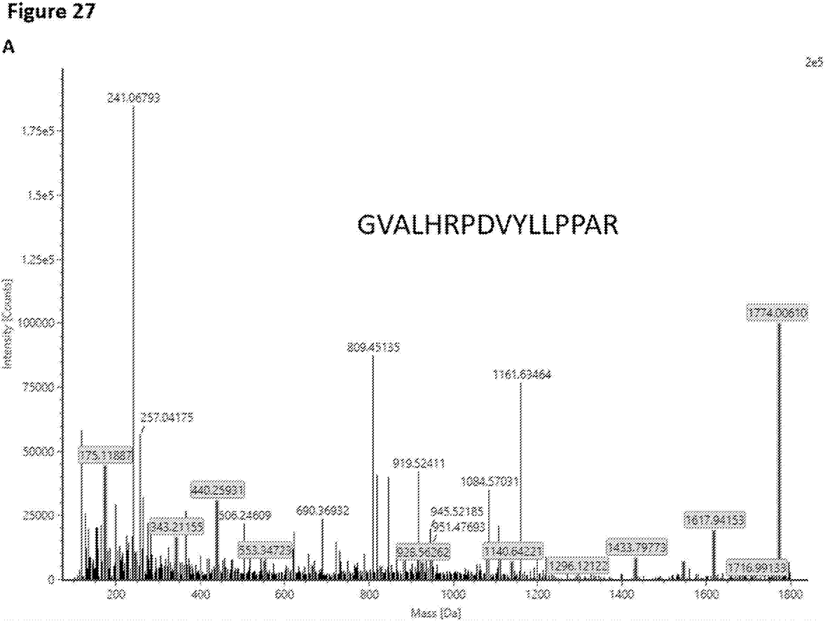

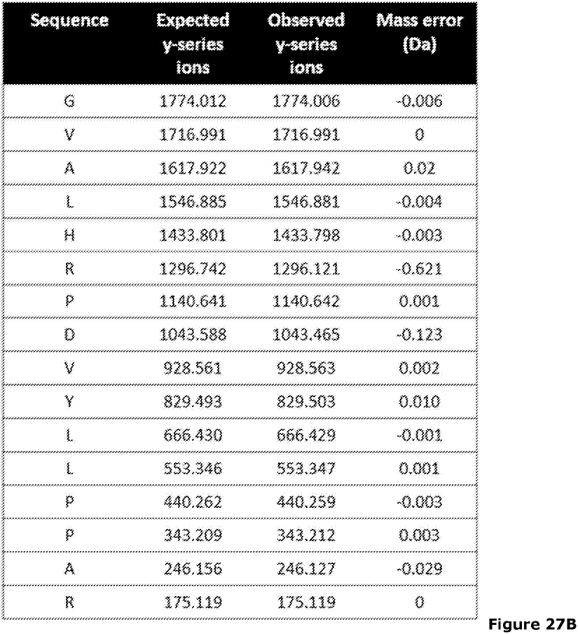

FIG. 27 Fragmentation spectrum corresponding to IgM peptide GVALHRPDVYLLPPAR within digested eluate of COVID-19 positive serum immunoprecipitated with beads conjugated with SARS-CoV-2 spike protein. (A) Fragmentation spectrum highlighting the C-terminal fragment ions (y-ion series). (B) Table of expected and observed y-ion series fragments.

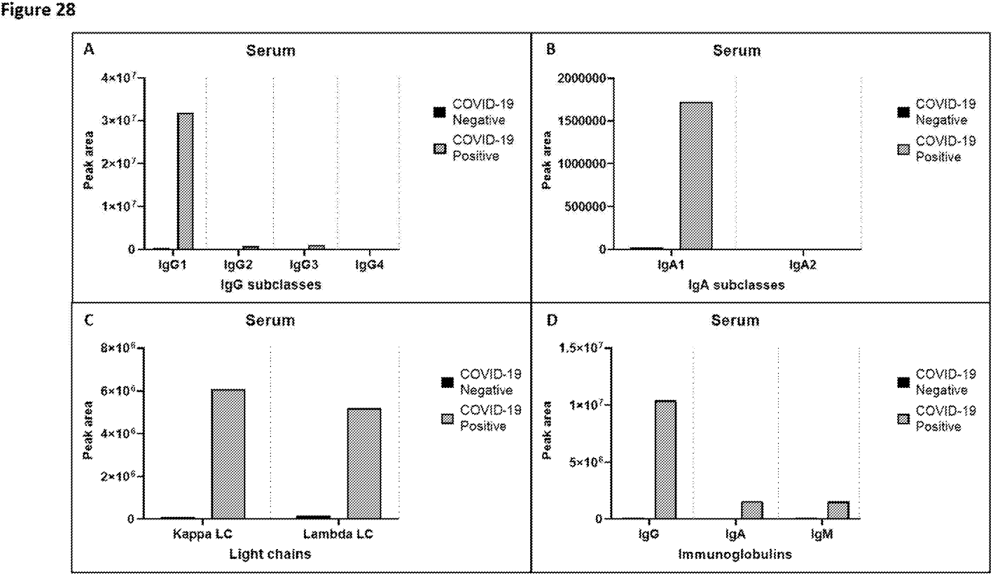

FIG. 28 Bar charts comparing peak areas of markers representing immunoglobulin isotypes IgG/IgA/IgM, IgG subclasses, IgA subclasses and light chains within digested eluates of negative and positive COVID-19 serum samples immunoprecipitated with beads conjugated with SARS-CoV-2 spike protein. Peak areas of (A) IgG subclasses, (B) IgA subclasses, (C) light chains, and (D) IgG/IgA/IgM immunoglobulins.

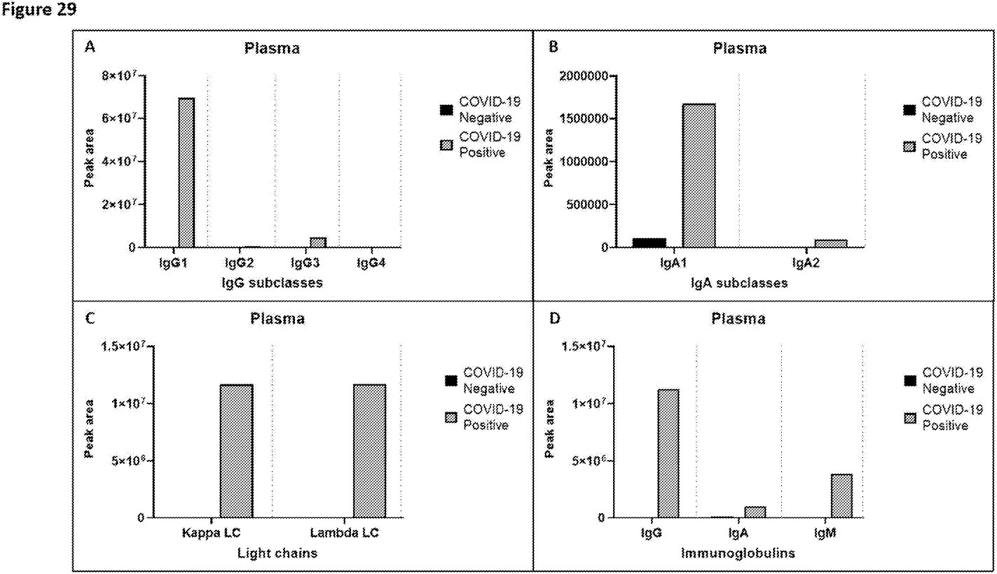

FIG. 29 Bar charts comparing peak areas of markers representing immunoglobulin isotypes IgG/IgA/IgM, IgG subclasses, IgA subclasses and light chains within digested eluates of negative and positive COVID-19 plasma samples immunoprecipitated with beads conjugated with SARS-CoV-2 spike protein. Peak areas of (A) IgG subclasses, (B) IgA subclasses, (C) light chains, and (D) IgG/IgA/IgM immunoglobulins.

The IgG classes and light chain types may be identified using techniques generally known in the art.

The spectrum obtained for different Ig classes and light chain types, using MALDI-TOF spectra are shown, for example, in FIG. 1. This utilises immunopurified immunoglobulins which have been dissociated with dithiothreitol as generally known in the art. This demonstrates that it is possible to identify the different heavy chain classes and light chain types. Method or performing such assays are shown in, for example WO2015/154052

FIG. 2 is an example from Ladwig et al, Supra, which demonstrates that it is also possible to determine subclasses of different immunoglobulins using mass spectrometry and quantifying them. Methods for carrying out that assay are described in that paper for example

Such techniques in the current invention are applied to antigen-specific immunoglobulins. A sample containing the immunoglobulin is contacted with an antigen attached to a substrate, such as a paramagnetic bead. The antigen-specific antibodies are then washed to remove non-specific binding, and eluted from the bead, prior to mass spectrometry.

FIGS. 3 (A and B) shows an example of a Coronavirus viral spike protein, attached to a paramagnetic bead. The upper panel shows that the activating and non-activating antibodies for receptor sites monoclonal antibody specifically binds to the viral spike protein bead, and is then eluted and detected via mass spectrometry as distinct peaks for immunoglobulin light chain and heavy chain. Normal serum and normal plasma immunoglobulins do not bind to the viral spike protein, so are washed off the bead, and are therefore not detected in the elution. The middle panel shows that pre-washing of the viral spike protein bead, does not substantially affect the immobilised protein, and still allows binding of the monoclonal antibody to it. The lower panel shows that if the viral spike protein is substituted for an a-IgG-specific antibody, then that monoclonal antibody binds to the bead. When such beads are incubated with normal plasma or normal serum, the IgG antibodies within the plasma or serum are detected, resulting in the overlapping broad lower peaks that are only observed in the lower panel.

Table 3 shows an example of a matrix of the typical amounts of different IgG subclasses and IgG that may be raised, in this example, against different viral proteins. Such a matrix may be expanded to include other classes, sub-types or light chains types. For example, IgE may be focussed on, for example, an IgE-related chronic disease such as allergic asthma or chronic urticaria might be being studies.

Such a matrix may also be converted into graphical format or other formats to allow comparison of the data between different immunoglobulin classes, subclasses, or light chain types, and different antigens. It may be automatically populated using a suitable computed implemented method.

To illustrate our approach we have analysed the immune response in four samples; 2 “diseased” individuals (PCR positive for Covid-19) and 2 “healthy” (tested negative for Covid-19) to 3 different infection related antigens; SARS CoV-2 Spike and Nucleocapsid protein and Pneumococcal cell wall polysaccharide (CWPS). Serum (sample 1 and 2) or plasma samples (3 and 4) of these 4 individuals were immunocaptured with these 3 antigen conjugated beads. Antibodies and antigen specific proteins captured by the beads are eluted, reduced and analysed by MALDI-TOF MS. In addition, antibodies and antigen specific proteins captured by the beads are eluted and analysed by LC-MS following tryptic digestion. Tryptic peptides for IgG1, IgG2, IgG3, IgG4, IgA1, IgA2, IgG, IgA, IgM heavy chains and kappa and lambda light chains were used to “profile” the immune response. For LC-MS analysis the international protein reference material Da470k was also run as a serum control.

### METHODS

### Immunoprecipitation and MALDI-TOF-MS

Antibodies and antigen-specific proteins were captured by antigen conjugated paramagnetic beads and eluted under reducing conditions to disassociate light chains from the heavy chains. Briefly, 50-150 µl of beads were washed three times with phosphate buffered saline, 0.1% Tween-20 (PBST). Diluted sample was added to the beads and incubated at room temperature with shaking for 30 mins. The beads were washed three times with PBST and then three more times with standard deionised water. 50 µL of 0.1% formic acid (LC-MS) or 5% acetic acid including reducing agent (MALDI-TOF) was used to elute the beads by incubation at room temperature with shaking for 15 mins. The elution was subsequently sandwich-spotted with MALDI matrix (α-Cyano-4-hydroxycinnamic acid) onto a MALDI-TOF target plate and dried. Mass spectra were acquired in positive ion mode on a Bruker Microflex MALDI-TOF-MS covering the m/z range of 5000 to 80,000 which includes the doubly charged ([M + 2H]2+, m/z 10900-12300) ions of the analyte (human kappa or lambda light chains). Light chain observed in the 2+ charge state can be sectioned into 3 regions specific to each light chain; Lambda (11200-11560 m/z), Kappa (11570-11850 m/z) and Heavy Kappa (11900-12400 m/z).

### LC-MS/MS and Digest Conditions

The eluate was transferred to a fresh microfuge tube and neutralised by the addition of 1 M triethylammonium bicarbonate (TEAB). The sample was then reduced with 200 mM tris(2-carboxyethyl)phosphine (TCEP), neutral pH for 30 mins at 60° C. at 1000 rpm before being cooled to room temperature. Alkylation was performed by the addition of 375 mM iodoacetamide and incubated for 30 mins at room temperature in the dark. Enzymatic digestion was carried out with 2.5 µL of 1 µg/µL trypsin and incubation of the sample for 2 hours at 37° C. at 1000 rpm. The digestion reaction was terminated with 1 µL of 100% formic acid. Sample volume was reduced using a vacuum concentrator at 60° C. on aqueous mode, prior to analysis by liquid-chromatography coupled electrospray ionisation mass spectrometry (LC-ESI-MS).

Samples were analysed on a Xevo G2-XS QToF mass spectrometer coupled to an ACQUITY I-Class UPLC system (Waters Ltd., Wilmslow, UK). 10 µL of the digested sample was injected onto an ACQUITY UPLC Peptide BEH C18, 130 Å, 1.7 µm, 2.1 × 150 mm column (Waters Ltd., Wilmslow, UK) maintained at 40° C. with a flow rate of 0.2 mL/min. A gradient from 0.1% (v/v) formic acid in water (A) to 0.1% formic acid (v/v) in acetonitrile (B) was employed. (gradient: 0-1 min, 1 % B; 1-60 min, 40% B; 60-70 min, 60% B; 70 min, 95% B; 70-80 min, 95% B; 80 min 1% B; 80-90 min 1% B). Capillary voltage was set to 1.5 kV with a 40 V cone voltage. Source temperature was set to 120° C. and desolvation temperature was set to 250° C. Cone gas glow was maintained at 50 L/h and desolvation gas flow at 600 L/h. MS^{E} was acquired over 90 mins scanning between 100-2000 m/z. Scans alternated between a low collisional energy of 6 eV for 0.5 sec and a high collisional energy ramp from 25 eV to 45 eV for another 0.5 sec. LockSpray™ was enabled and Leucine Enkephalin was measured for 0.25 seconds every minute at 3 kV capillary voltage and 30 V cone voltage. The ions monitored for MS^{E} relative quantitation are outlined in Table 5. The fragmentation spectrum of each individual immune-marker peptide are shown in FIGS. 17 to 27.

### RESULTS

### Maldi-tof

The overall antibody response against antigen conjugated beads was measured as peak intensity (a.u.) and characterised by peak distribution from a MALDI-MS data. Most individuals tested against the bacterial polysaccharide present a low level of natural antibody response (FIG. 4). This excludes Individual 3 (Covid-19 positive), who had a significantly high response to the bacterial antigen suggesting a recent bacterial infection. Overall, the light chain distribution observed against the bacterial antigen was predominantly polyclonal and oligoclonal with a bias towards kappa light chain usage, particularly heavy kappa. The same individuals were tested against the viral SARS-Cov-2 spike protein (FIG. 4). Covid-19 positive individuals (2 and 4) displayed a high antibody response against the spike protein, consisting of an underlying polyclonal light chain distribution (smooth peaks) with oligoclonal light chains (sharp peaks). The “healthy” Covid-19 negative individuals (2 and 4) present with a baseline antibody response. Subsequently, we compared the immune response from the SARS-Cov-2 nucleocapsid protein to the spike protein to characterise the response to antigens that differ in size, structure, function, and localisation (FIG. 5). As observed with the spike protein, the antibody response from Covid-19 positive individuals (2 and 4) also elicits a high response against the nucleocapsid protein. The nucleocapsid response largely consisted of lambda light chains with few oligoclonal peaks (FIG. 5). The relative response from “healthy” Covid-19 negative individuals (1 and 3) against nucleocapsid protein is close to baseline (FIG. 5). The κ:λ ratio of all 4 samples against the bacterial Pneumococcal Cell wall polysaccharide had a high κ:λ ratio suggesting a kappa light-chain bias (Table 4). The κ:λ ratio for response against the viral protein antigens on bead (SAR-Cov-2 Spike and Nucleocapsid protein) were variable with no evidence for a bias. The two Covid-19 positive individuals (2 and 4) show a response against Nucleocapsid protein, with κ:λ ratio of <1 suggesting the response is dominated by lambda light chain usage (Table 4).

In summary, the MALDI-TOF analysis has provided an overview of the antibody response to bacterial and viral antigens and indicated differences in quantity and quality of the immune response between these 4 individuals.

### Lc- Ms/ms

An immune-marker tryptic- peptide that was specific (diagnostic) for the immunoglobulin being detected was chosen for each human immunoglobulin (IgG1, IgG2, IgG3, IgG4, IgA1, IgA2, IgG, IgA, IgM heavy chains and kappa and lambda light chains, (Table 5). The amino acid sequence and identity of these peptides was confirmed using the respective fragmentation ion spectrum as indicated in FIGS. 17-27. In each case the MS spectrum (A) is accompanied by the fragmentation ion table (B). These peptides were selected based on their length, relative abundance, and that they only occurred once in the polypeptide sequence of the protein.

These peptides were used to profile the immune response in four human samples; 2 “disease-state” individuals (PCR positive for Covid-19) and 2 “healthy” (tested negative for Covid-19) to 3 different microbial infection related antigens; SARS CoV-2 Spike and Nucleocapsid protein and Pneumococcal CWPS. In each case the extracted ion chromatogram was obtained to identify the presence of that peptide, and the area-under-the-peak was calculated and used for as a surrogate marker for the comparative measurement of the intact immunoglobulin from which it originated. For example the extracted ion chromatograms obtained for these 4 human clinical samples against the SARS-Cov-2 Spike protein is shown for IgG1 (FIG. 6), IgG2 (FIG. 7), IgG3 (FIG. 8), IgG4 (FIG. 9), IgA1 (FIG. 10), IgA2 (FIG. 11), and kappa (FIG. 12) and lambda light chains (FIG. 13). Additionally, peptide markers for total IgG (FIG. 14), total IgA (FIG. 15) and total IgM (FIG. 16) are also obtained. For comparison the international protein reference material ERM-DA470k was independently digested and run as a serum immunoglobulin control. The relative abundance, seen in FIGS. 6-16, can be compared and used to profile the antibody response between healthy and disease-state samples This is shown graphically for serum (FIG. 28) and plasma (FIG. 29) matrices. These have been sorted for easy comparison into IgG (FIGS. 28A and 29A) and IgA subclasses (FIGS. 28B and 29B), immunoglobulin light chains (FIGS. 28C and 29C) and total immunoglobulin IgG, IgA, and IgM (FIGS. 28D and 29D). The immune response of the Covid-positive samples is much greater than that of the Covid-negative samples which is dominated by an IgG response. In terms of subclass response both IgG1 and IgA1 are the dominant subclasses present. The light chain usage is approximately the same between kappa and lambda. This mirrors that observed using the MALDI-TOF platform (Table 4).

Equivalent analyses of the immunoglobulins immuno-captured by the Nucleocapsid protein and Pneumococcal CWPS beads was also produced. This data is combined with that of the Spike protein in Table 6 and expressed as a relative abundance profile. As expected the antibody immune response to the nucleocapsid protein antigen was lower in the Covid- negative (healthy) patients compared to the positive (disease-state) ones. In contrast to the two SARS-CoV-2 antigens, the immune response to Pneumococcal CWPS was more balanced between Covid positive and negative patients but was strongly biased to an IgG2 and kappa-light chain response. This also supported the results observed using MALDI-TOF-MS.

In summary, the tryptic-peptide LC-MS/MS analysis has provided a detailed overview of the antibody response to bacterial and viral antigens and allowed the antibody immune response between these 4 individuals to be profiled.

TABLE 1

| General | IgG1 | IgG2 | IgG3 | IgG4 |
|---|---|---|---|---|
| Molecular mass (kD) | 146 | 146 | 170 | 146 |
| Amino acids in hinge region | 15 | 12 | 62 | 12 |
| Inter-heavy chain disulfide bonds | 2 | 4 | 11 | 2 |
| Mean adult serum level (g/l) | 6.98 | 3.8 | 0.51 | 0.56 |
| Relative abundance (%) | 60 | 32 | 4 | 4 |
| Half-life (days) | 21 | 21 | 7/~21 | 21 |
| Placental transfer | + + + + | ++ | ++/++++ | +++ |
| Antibody response to: | | | | |
| Proteins | ++ | +/- | ++ | ++ |
| Polysaccharides | + | +++ | +/- | +/- |
| Allergens | + | (-) | (-) | ++ |
| Complement activation | | | | |
| C1q binding | ++ | + | +++ | - |
| Fc receptors | | | | |
| FcyRI | +++ | | ++++ | ++ |
| FcyRIIa_{H131} | +++ | ++ | ++++ | ++ |
| FcyRIIa_{R131} | +++ | + | ++++ | ++ |
| FcyRIIb/c | + | | ++ | + |
| FcyRIIIa_{F158} | ++ | | ++++ | - |
| FcyRIIIa_{V158} | +++ | + | ++++ | ++ |
| FcyRIIIb | +++ | | + + + + | - |
| FcRn (at pH <6.5) | +++ | +++ | ++/+++ | +++ |

TABLE 2

| | | |
|---|---|---|
| Protein | Peptide | Transitions |
| IgG1 | GPSVFPLAPSSK | y8-y10 |
| IgG2 | GLPAPIEK | y4-y6 |
| IgG3 | WYVDGVEVHNAK | y6, y8, y9 |
| IgG4 | TTPPVLDSDGSFFLYSR | y8,y10,y12 |
| IgA1 | TPLTATLSK | y5-y7 |
| IgA2 | DASGATFTWTPSSGK* | y7-y10 |
| IgA1-2 | WLQGSQELPR | y6-y8 |
| IgM | DGFFGNPR | y4-y6 |
| κ LC | TVAAPSVFIFPPSDEQLK* | y8,y9,y11 |
| λ LC | AGVETTTPSK | y5-y7 |
| IgD | EPAAQAPVK | y5-y7 |
| IgE | GSGFFVFSR* | y5-y7 |

TABLE 3

| | IgG1 (mg/L) | IgG2 (mg/L) | IgG3 (mg/L) | IgG4 (mg/L) | IgG (mg/L) | IgA1 (mg/L) | IgA2 (mg/L) | IgA (mg/L) | IgM (mg/L) | IgD (mg/L) | IgE (mg/L) |
|---|---|---|---|---|---|---|---|---|---|---|---|
| Spike 1 | 10 | 1 | 100 | 0.1 | 111 | | | | | | |
| Spike 2 | 11 | 1 | 100 | 0.1 | 112 | | | | | | |
| membrane | 30 | 5 | 300 | 0.1 | 335 | | | | | | |
| Envelope | 40 | 1 | 500 | 0 | 541 | | | | | | |
| Nucleocapsid | 0.1 | 0.1 | 500 | 0 | 500 | | | | | | |
| Whole Virus | 100 | 20 | 2000 | <1 | 2120 | | | | | | |
| Virus lysate | 300 | 40 | 2000 | <1 | 2340 | | | | | | |

TABLE 4

Lambda (κ:λ) ratio of antibody response

| Antigen conjugated on bead | Samples Sample Type | | Sample description* | K:L Ratio |
|---|---|---|---|---|
| Bacterial (Pneumococcal Cell wall polysaccharide) | Individual 1 | Serum | Healthy | 4.11 |
| | Individual 2 | Serum | Diseased | 4.31 |
| | Individual 3 | Plasma | Healthy | 5.85 |
| | Individual 4 | Plasma | Diseased | 5.81 |
| Viral (SARS-CoV-2 Spike protein) | Individual 1 | Serum | Healthy | N/D |
| | Individual 2 | Serum | Diseased | 1.76 |
| | Individual 3 | Plasma | Healthy | N/D |
| | Individual 4 | Plasma | Diseased | 2.43 |
| Viral (SARS-CoV-2 Nucleocapsid protein) | Individual 1 | Serum | Healthy | N/D |
| | Individual 2 | Serum | Diseased | 0.67 |
| | Individual 3 | Plasma | Healthy | N/D |
| | Individual 4 | Plasma | Diseased | 0.56 |

* Sample description of “Healthy” refers to individuals tested PCR negative for Covid-19 and “Diseased” were individuals that test PCR positive.

Antigen-specific immunoglobulins were captured and eluted under reducing conditions to disassociate light chains from the heavy chains. Eluents were analysed by MALDI-Mass spectrometry, focusing on the distribution of light chains in the 2+ charge state (11000-12500 m/z). light chain observed in the 2+ charge state can be sectioned into 3 regions specific to each light chain; Lambda (11200-11500 m/z), Kappa (11600-11850 m/z) and Heavy Kappa (11900-12200 m/z). Area under the peak for lambda and total kappa lambda was used to calculate the κ:λ ratio for all antibody response using a open source mmass software.

TABLE 5

List of markers used for immunoglobulin isotypes IgG, IgA and IgM, IgG subclasses, IgA subclasses, and light chains

| Marker | Peptide | Retention time (min) | Precursor ion (m/z) |
|---|---|---|---|
| IgG1 | TPEVTC_{(CAM)}VVVDVSHEDPEVK | 30.10 | 713.69 |
| IgG2 | GLPAPIEK | 21.14 | 412.75 |
| IgG3 | TPEVTC_{(CAM)}VVVDVSHEDPEVQFK | 33.98 | 805.39 |
| IgG4 | GLPSSIEK | 18.52 | 415.74 |
| IgA1 | DASGVTFTWTPSSGK | 30.12 | 770.87 |
| IgA2 | DASGATFTWTPSSGK | 27.04 | 756.85 |
| Kappa LC | SGTASVVC_{(CAM)}LLNNFYPR | 43.02 | 899.45 |
| Lambda LC | YAASSYLSLTPEQWK | 35.15 | 872.43 |
| IgG | DTLMISR | 21.35 | 418.22 |
| IgA | SGNTFRPEVHLLPPPSEELALNELVTLTC_{(CAM)}LAR | 48.82 | 894.22 |
| IgM | GVALHRPDVYLLPPAR | 28.93 | 444.26 |

Retention time (minutes) and Precursor ion (mass: charge [m/z]) listed for each marker peptide used for relative quantitation. Peak identified within a retention tolerance of ± 0.1 minute and precursor ion tolerance of ± 0.1 m/z.

TABLE 6

Relative quantitation of immunoglobulins IgG/IgA/IgM, IgG subclasses, IgA subclasses and light chains within digested eluates of serum and plasma samples negative or positive for COVID-19 against beads conjugated with SARS-CoV-2 spike protein, SARS-CoV-2 nucleocapaid protein or pneumococcal cell wall polysaccharide (Streptococcus pneumoniae)

| | ERM-DA470k | SARS-CoV-2 spike protein | | | | SARS-CoV-2 nucleocapsid protein | | | | Pneumococcal cell wall polysaccharide | | | |
|---|---|---|---|---|---|---|---|---|---|---|---|---|---|
| | | Serum | | Plasma | | Serum | | Plasma | | Serum | | Plasma | |
| | | Negative | Positive | Negative | Positive | Negative | Positive | Negative Positive | | Negative | Positive | Negative | Positive |
| IgG1 | 70.2% | 82.3% | 94.4% | 77.3% | 92.6% | 77.9% | 88.0% | 82.3% | 93.1% | 17.8% | 30.1% | 9.9% | 7.2% |
| IgG2 | 22.9% | 15.0% | 2.4% | 15.8% | 1.0% | 11.9% | 7.6% | 15.0% | 1.5% | 79.7% | 59.3% | 39.3% | 91.7% |
| IgG3 | 3.8% | 2.7% | 3.2% | 6.5% | 6.4% | 7.3% | 3.6% | 2.7% | 5.3% | 1.7% | 8.8% | 1.3% | 0.6% |
| IgG4 | 3.1% | 0.0% | 0.0% | 0.5% | 0.0% | 3.3% | 0.7% | 0.0% | 0.1% | 0.9% | 1.6% | 0.1% | 0.4% |
| IgA1 | 90.8% | 83.8% | 99.3% | 98.2% | 94.6% | 88.9% | 93.4% | 83.8% | 91.0% | 87.1% | 87.4% | 86.8% | 32.2% |
| IgA2 | 9.2% | 15.2% | 0.7% | 1.9% | 5.2% | 11.1% | 6.6% | 16.2% | 9.0% | 12.9% | 12.6% | 13.2% | 17.8% |
| Kappa LC | 60.8% | 38.0% | 53.9% | 49.4% | 50.0% | 57.6% | 31.5% | 45.5% | 18.0% | 73.4% | 65.2% | 79.4% | 78.6% |
| Lambda LC | 39.2% | 62.0% | 46.1% | 50.6% | 50.0% | 42.4% | 68.5% | 54.5% | 82.0% | 26.6% | 34.8% | 20.6% | 21.4% |
| K:L | 1.55 | 0.61 | 1.17 | 0.98 | 1.00 | 1.55 | 0.46 | 0.83 | 0.22 | 2.76 | 1.87 | 3.65 | 3.67 |
| IgG | 72.1% | 47.7% | 77.0% | 22.8% | 69 .6% | 11.0% | 59.5% | 1.1% | 70.4% | 2.6% | 2.2% | 50.2% | 50.5% |
| IgA | 19.1% | 8.1% | 11.7% | 52.8% | 6.2% | 21.0% | 7.2% | 1.3% | 5.7% | 0.1% | 7.0% | 4.4% | 2.4% |
| IgM | 8.8% | 44.2% | 11.4% | 14.3% | 24.2% | 68.0% | 33.3% | 97.6% | 23.9% | 97.2% | 90.8% | 45.4% | 47.2% |

Percentage compositions were calculated from the peak area of the monoisotopic peak of the marker peptides. Distribution of immunoglobulin components are subdivided into five categories: IgG subclasses, IgA subclasses, light chains (LC), light chain (kappa to lambda) ratio, and immunoglobulin isotypes: IgG, IgA and IgM. The most abundant component for each category is highlighted in grey.

1. A method of identifying or characterising an immune response in a subject comprising:
(a) contacting a sample containing immunoglobulins from the subject with at least one antigen immobilised on a support;
(b) washing unbound, non-antigen specific immunoglobulins from the support to leave antigen-specific immunoglobulins bound to the antigen on the support;
(c) optionally eluting the antigen-specific immunoglobulins from the antigen on the support; and
(d) subjecting the antigen-specific immunoglobulins to mass spectrometry to identify two or more different antigen specific immunoglobulin classes, subclasses and/or light chain types.

2. The method according to claim 1, wherein the sample is contacted with at least two different antigens, wherein each antigen on a different support or a different portion of the same support, and wherein the sample is optionally separated into at least two aliquots, and each aliquot is contacted with a different antigen bound to a different support.

3-4. (canceled)

5. The method according to claim 1, wherein
(a) at least a portion of the antigen-specific immunoglobulins are subjected to a proteolytic digestion prior subjecting the digested antigen-specific immunoglobulins to the mass spectrometry;
(b) at least a portion of the antigen-specific immunoglobulins are not subjected to a proteolytic digestions prior to subjecting the antigen-specific immunoglobulins to the mass spectrometry; or
(c) at least a portion of the antigen-specific immunoglobulins are dissociated with at least one reducing agent and/or denaturing agent to separate light chains bound to heavy chains prior to subjecting the separated immunoglobulins to the mass spectrometry.

6-7. (canceled)

8. The method according to claim 1, wherein
(a) the relative amount of the two or more of the different antigen specific immunoglobulin classes, subclasses and/or light chain types are compared to each other, or the amount of each of two or more of the different antigen specific immunoglobulin classes, subclasses and/or light chain types in the sample are determined; or
(b) the amount of one or more different antigen specific immunoglobulin classes, subclasses and/or light chain types in the sample is quantitated.

9. (canceled)

10. The method according to claim 1,
(a) wherein the immunoglobulin classes are selected from IgG, IgA, IgM, IgD and IgE;
(b) wherein the immunoglobulin subclasses are selected from IgG1, IgG2, IgG3, IgG4, IgA1 and IgA2;
(c) wherein the immunoglobulin light chains are selected from lambda light chains and kappa light chains;
(d) wherein the ratio of the relative amount of lambda : kappa light chains in the sample is determined;
(e) wherein the method additionally comprises identifying one or more of a) J-chains bound to IgA and/or IgM and/or b) CD5L-bound to IgM;
(f) wherein the immunoglobulins in the sample are purified or enriched, prior to contacting the antigen bound to the support; or
(g) wherein at least a portion of the IgG in the sample is removed prior to contacting the remaining immunoglobulins with the antigen bound to the support.

11-16. (canceled)

17. The method according to claim 1, wherein, in step (c) antigen-specific immunoglobulins having lower antigen binding specificity are eluted from the antigen bound to the support prior to those having a higher antigen binding specificity.

18. The method according to claim 17, wherein, the or each antigen is an antigen from a virus, a bacterium, an archaebacterium, a fungus, a protozoan, a helminth, an autoimmune antigen, a cancer antigen or an antigen capable of inducing an allergic reaction in the subject.

19. The method according to claim 1, wherein at least a portion of the IgG in the sample is removed prior to contacting the remaining immunoglobulins with the antigen bound to the support and wherein the virus is a Coronavirus.

20. The method according to claim 17, wherein the virus antigen is at least an antigenic portion of a viral envelope protein, a capsid protein, an enzyme or haemagglutinin.

21. The method according to claim 17, wherein at least a portion of the IgG in the sample is removed prior to contacting the remaining immunoglobulins with the antigen bound to the support and wherein the bacterial antigen is at least one antigen portion of a cellular antigen, a flagella antigen, a somatic antigen, a virulence antigen, a fimbrial antigen or a toxoid.

22. The method according to claim 1, wherein the subject is a fish, a mammal, a bird, or a reptile, wherein the mammal is optionally selected from a human, a non-human ape, a monkey, a horse, a sheep, a camelid, a goat, a cow, a dog, a cat, or a rodent.

23. (canceled)

24. The method according to claim 17,
(a) wherein the sample is a sample of biological fluid, typically blood, serum, plasma, cerebrospinal fluid, urine, tear, sputum, lavage fluid, or saliva;
(b) wherein the support is selected from paramagnetic beads and a MALDI-TOF target;
(c) wherein one or more additional indicators of the immune response in the subject is additionally determined;
(d) wherein the mass spectrometry is Liquid Chromatography Mass Spectrometry or MALDI-TOF mass spectrometry; or
(e) further comprising adding a predetermined amount of a calibrator to the sample.

25. (canceled)

26. The method according to claim 17, wherein an ionisation control is added to the sample, prior to carrying out the mass spectrometry.

27-28. (canceled)

29. The method according to claim 1, and further comprising producing a matrix to characterise an immune response in the subject by measuring of an amount of two or more two or more of the different antigen specific immunoglobulin classes, subclasses and/or light chain types compared to two or more different antigens.

30. (canceled)

31. The method according to claim 1, and further comprising
(a) identifying an the-immune status of the subject;
(b) characterising the immune response in the subject to a pathogen, allergen or other antigen;
(c) selecting one or more vaccine targets, and determining severity or progression of a condition caused by a pathogen;
(d) characterising an autoimmune response in the subject;
(e) characterising an allergic response in the subject;
(f) monitoring the immune response or progression of a disease in the subject; or
(g) selecting a monoclonal antibody class or monoclonal antibody characteristics.

32-37. (canceled)

38. The method according to claim 1, whereby the neutralizing capacity of an immunoglobulin is determined by measuring molecules in the sample buffer indicative of neutralization of the bound antigen.

39. The method according to claim 1, to identify activating and non-activating antibodies for receptor sites.

40. A computer implemented method for identifying or characterising an immune response in a subject, comprising comparing a mass spectrum obtained for a first antigen specific immunoglobulin class, subclass and/or light chain type with a mass spectrum for a second antigen specific immunoglobulin class, subclass and/or light chain type, wherein the mass spectrum is obtained by a method according to claim 1.

41. The computer implemented method according to claim 40, wherein the computer comprises a computer processor and a computer memory.

42. An apparatus for identifying or characterising an immune response in a subject by a method according to claim 1, comprising the use of a computer implemented method comprising comparing a mass spectrum obtained for a first antigen specific immunoglobulin class, subclass and/or light chain type with a mass spectrum for a second antigen specific immunoglobulin class, subclass and/or light chain type, wherein the apparatus optionally includes a mass spectrometer.

43-44. (canceled)


SEQUENCE LISTING (22 sequences)

SEQ ID NO: 1 - protein, 12 aa, Homo sapiens
GPSVFPLAPSSK

SEQ ID NO: 2 - protein, 8 aa, Homo sapiens
GLPAPIEK

SEQ ID NO: 3 - protein, 12 aa, Homo sapiens
WYVDGVEVHNAK

SEQ ID NO: 4 - protein, 17 aa, Homo sapiens
TTPPVLDSDGSFFLYSR

SEQ ID NO: 5 - protein, 9 aa, Homo sapiens
TPLTATLSK

SEQ ID NO: 6 - protein, 15 aa, Homo sapiens
DASGATFTWTPSSGK

SEQ ID NO: 7 - protein, 10 aa, Homo sapiens
WLQGSQELPR

SEQ ID NO: 8 - protein, 8 aa, Homo sapiens
DGFFGNPR

SEQ ID NO: 9 - protein, 18 aa, Homo sapiens
TVAAPSVFIFPPSDEQLK

SEQ ID NO: 10 - protein, 10 aa, Homo sapiens
AGVETTTPSK

SEQ ID NO: 11 - protein, 9 aa, Homo sapiens
EPAAQAPVK

SEQ ID NO: 12 - protein, 9 aa, Homo sapiens
GSGFFVFSR

SEQ ID NO: 13 - protein, 19 aa, Homo sapiens
TPEVTCVVVDVSHEDPEVK

SEQ ID NO: 14 - protein, 21 aa, Homo sapiens
TPEVTCVVVDVSHEDPEVQFK

SEQ ID NO: 15 - protein, 8 aa, Homo sapiens
GLPSSIEK

SEQ ID NO: 16 - protein, 15 aa, Homo sapiens
DASGVTFTWTPSSGK

SEQ ID NO: 17 - protein, 15 aa, Homo sapiens
DASGATFTWTPSSGK

SEQ ID NO: 18 - protein, 16 aa, Homo sapiens
SGTASVVCLLNNFYPR

SEQ ID NO: 19 - protein, 15 aa, Homo sapiens
YAASSYLSLTPEQWK

SEQ ID NO: 20 - protein, 7 aa, Homo sapiens
DTLMISR

SEQ ID NO: 21 - protein, 32 aa, Homo sapiens
SGNTFRPEVHLLPPPSEELALNELVTLTCLAR

SEQ ID NO: 22 - protein, 16 aa, Homo sapiens
GVALHRPDVYLLPPAR